SEQUENCE LISTING (2 sequences)

SEQ ID NO: 1 - protein, 9 aa, Homo sapiens
KVPRNQDWL

SEQ ID NO: 2 - protein, 9 aa, Mus musculus
EGSRNQDWL

(19) United States
(12) Patent Application Publication
(10) Pub. No.: US 2026/0034216 A1
(43) Pub. Date: Feb. 5, 2026

(54) COMPOSITIONS AND METHODS FOR IMMUNE CELL MODULATION IN ADOPTIVE CELL THERAPY

(71) Applicants: The Johns Hopkins University, Baltimore, MD (US); Iyuda Therapeutics, Inc., Owings Mills, MD (US)

(72) Inventors: Ephraim Fuchs, Baltimore, MD (US); Robert E. Tennant, Owings Mills, MD (US)

(21) Appl. No.: 19/093,011

(22) Filed: Mar. 27, 2025

Related U.S. Application Data
(63) Continuation of application No. PCT/US2023/034036, filed on Sep. 28, 2023.
(60) Provisional application No. 63/377,696, filed on Sep. 29, 2022.

(51) Int. Cl. A61K 40/11; A61K 40/31; A61K 40/32; A61K 40/42

(52) U.S. Cl. CPC A61K 40/11; A61K 40/31; A61K 40/32; A61K 40/4211; A61K 40/4224

(57) ABSTRACT

The disclosure relates to adoptive cell therapy compositions including a population of isolated immune cells that are obtained from a donor subject. The immune cells can be modified to suppress Bruton's tyrosine kinase (BTK), interleukin-2-inducible T cell kinase (ITK), delta isoform of phosphoinositide 3-kinase (PI3Kδ), helios, blimp1, SOCS1, GATA3, IL-10, STAT3, TOX, CD25, foxp3, Ezh2, TGF-beta Receptor II, LAG-3, PD-1, TNF-alpha, or combinations thereof. The immune cells are optionally depleted of CD8+ T cells by about 10-fold or greater relative to un-depleted leukocytes.

### CROSS-REFERENCE TO RELATED APPLICATIONS

This application is a continuation application of International Application No. PCT/US2023/034036 filed Sep. 28, 2023, now pending; which claims the benefit under 35 USC § 119(e) to U.S. Application Ser. No. 63/377,696 filed Sep. 29, 2022, now expired. The disclosure of each of the prior applications is considered part of and is incorporated by reference in the disclosure of this application.

### INCORPORATION OF SEQUENCE LISTING

The material in the accompanying sequence listing is hereby incorporated by reference into this application. The accompanying sequence listing xml file, named JHU4560-1_ST26.xml, was created on Mar. 6, 2025 and is 2,850 bytes in size.

### BACKGROUND OF THE INVENTION

### Field of the Invention

The present disclosure relates generally to adoptive cell therapy and more specifically to a population of isolated immune cells that are obtained from a donor subject.

### Background Information

Cancer immunotherapy has emerged as a promising cancer treatment modality. The goal of cancer immunotherapy is to harness the immune system for selective destruction of cancer cells while leaving normal tissues unharmed.

Cytotoxic CD8+ T cells are a major population of immune cells that control and clear tumor cells. Zhang et al., (2020), Front. Cell Dev. Biol., 8:17. However, T cells are not as effective against cancer as expected. Due to immunologic tolerance and immunosuppressive mechanisms, CD8+ T cells are often sub-optimally primed and for this reason and others can fail to be effective or enter a dysfunctional state known as T cell exhaustion. T cell exhaustion leads to attenuated effector function whereby cytotoxic CD8+ T cells are impaired in their ability to kill cancer cells, leading to tumor progression.

There is great interest in developing methods to revive or replace exhausted T cells to restore the anti-tumor immune response. However, challenges still remain to develop effective methods for reviving exhausted T cells.

Adoptive cell therapies have seen some clinical success but have not reached their full potential. Chimeric antigen receptor-modified T cell (CAR-T) therapies targeting CD19 or BCMA have been approved for B cell cancers and multiple myeloma, respectively, but their effectiveness is limited by lack of persistence and antigen escape. CAR-T therapy has overall not been successfully employed against solid tumors, in part as a result of the immunosuppressive tumor microenvironment along with physical barriers and the heterogeneity of solid tumors. CAR-T therapy is also associated with toxicity including cytokine release syndrome (CRS), neurotoxicity, and on target off tumor effects (Siegler et al., Front Immunol (2020) 11:1973). Cell therapies focusing on TCRs or other immune cell receptors have grown in popularity in recent years, and many such programs are in clinical trials. TCR therapies, like CAR-T therapies, are susceptible to loss of T cell persistence and antigen escape. Additional challenges to TCR based therapy include their restriction to MHC presented antigens, the limited availability of tumor-specific T cells and deficiencies in antigen processing or major histocompatibility complex (MHC) expression in cancer cells.

Tumor infiltrating lymphocytes (TILs) have been more successful in treating solid tumors such as metastatic melanoma and cervical cancer than CAR-T therapy or exogenous TCR based treatments. This is thought to be in part a result of TIL populations having multiple TCRs capable of recognizing tumor antigens, lessening the issue of tumor heterogeneity. However, TILs must overcome the immunosuppressive microenvironment and physical barriers associated with solid tumors.

CAR and TCR based cell therapies suffer from antigen escape and limited persistence even in the indications in which they have seen the most success, and they have largely been ineffective against solid tumors. They also have the potential to cause significant toxicity.

As such, there is a substantial need for adoptive cell therapies that have improved effectiveness and persistence while avoiding or minimizing toxicity.

### SUMMARY OF THE INVENTION

The disclosure relates to an adoptive cell therapy composition. The adoptive cell therapy composition comprises a population of isolated immune cells that are obtained from a donor subject. The immune cells can be modified to suppress Bruton's tyrosine kinase (BTK), interleukin-2-inducible T cell kinase (ITK), delta isoform of phosphoinositide 3-kinase (PI3Kδ), helios, blimp1, SOCS1, GATA3, IL-10, STAT3, TOX, CD25, foxp3, Ezh2, TGF-beta Receptor II, LAG-3, PD-1, TNF-alpha, or combinations thereof. The immune cells are optionally depleted of CD8+ T cells by about 10-fold or greater relative to un-depleted leukocytes.

The adoptive cell therapy composition can comprise one or more T cells. For example, the adoptive cell therapy composition can comprise tumor infiltrating lymphocytes (TILs), CAR-T, or T-cell receptor (“TCR”)-transduced T cells.

The adoptive cell therapy can comprise an immune cell that comprises an antigen binding protein. The antigen binding protein can have specificity for an antigen selected from the group consisting of CD19, CD123, CD22, CD30, CD171, CS-1, CLL-1 (CLECL1), CD33, EGFRvIII, GD2, GD3, BCMA, Tn Ag, PSMA, ROR1, FLT3, TAG72, CD38, CD44v6, CEA, EPCAM, B7H3, KIT, IL-13Ra2, Mesothelin, IL-11Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, FAP, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, TSHR, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, legumain, HPV E6, E7, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, fibronectin EDB (EDB-FN), 5T4 oncofetal antigen, and IGLL1.

At least a portion of the immune cells can be differentiated to Th1 CD4+ T cells. The immune cells can be biased toward Th1 CD4+ T cell differentiation by inhibition of BTK. The immune cells can be biased toward Th1 CD4+ T cell differentiation by inhibition of ITK. The immune cells can be biased toward Th1 CD4+ T cell differentiation T cells by inhibition of PI3Kδ. The immune cells can be biased toward Th1 CD4+ T cell differentiation by inhibition of Foxp3. The immune cells can be biased toward Th1 CD4+ T cell differentiation by inhibition of GATA3. The immune cells can be biased toward Th1 CD4+ T cell differentiation by inhibition of STAT3. The immune cells can be biased toward Th1 CD4+ T cell differentiation by inhibition of CD25. The immune cells can be biased toward Th1 CD4+ T cell differentiation by inhibition of Ezh2. The immune cells can be biased toward Th1 CD4+ T cell differentiation by inhibition of both BTK and ITK. The immune cells can be biased toward Th1 CD4+ T cell differentiation and against regulatory T cell differentiation by inhibition of both BTK and PI3Kδ. The immune cells can be biased toward Th1 CD4+ T cell differentiation by inhibition and against regulatory T cell differentiation of both ITK and PI3Kδ. The T cells can be biased toward Th1 CD4+ T cell differentiation by inhibition and against regulatory T cell differentiation of BTK, ITK and PI3Kδ.

BTK, ITK and PI3Kδ, helios, blimp1, SOCS1, GATA3, IL-10, STAT3, TOX, CD25, TGF-beta Receptor II, LAG-3, PD-1, TNF-alpha, foxp3, or Ezh2 can be inhibited with an inhibitor or by genetic modification. The ITK inhibitor, the BTK inhibitor, the PI3Kδ inhibitor, the Helios inhibitor, the Blimp1 inhibitor, the SOCS1 inhibitor, the Foxp3 inhibitor, the TGF-beta Receptor II inhibitor, the LAG-3 inhibitor, the PD-1 inhibitor, the TNF-alpha inhibitor, the GATA3 inhibitor, the IL-10 inhibitor, the STAT3 inhibitor, the CD25 inhibitor, the Ezh2 inhibitor, or the TOX inhibitor can be a small molecule, a small interfering RNA (siRNA), or short hairpin RNA (shRNA).

BTK, ITK, PI3Kδ, Helios, Blimp1, SOCS1, Foxp3, GATA3, IL-10, TGF-beta Receptor II, LAG-3, PD-1, TNF-alpha, STAT3, Ezh2, CD25, or TOX can be inhibited by deleting the BTK gene, the ITK gene, the PI3Kδ gene, the Helios gene, the Blimp1 gene, the SOCS1 gene, the Foxp3 gene, the TGF-beta Receptor II gene, the LAG-3 gene, the PD-1 gene, the TNF-alpha gene, the GATA3 gene, the IL-10 gene, the STAT3 gene, the Ezh2 gene, the CD25 gene, or the TOX gene from the genome.

The BTK gene, the ITK gene, the PI3Kδ gene, the Helios gene, the Blimp1 gene, the SOCS1 gene, the Foxp3 gene, the TGF-beta Receptor II gene, the LAG-3 gene, the PD-1 gene, the TNF-alpha gene, the GATA3 gene, the IL-10 gene, the STAT3 gene, the Ezh2 gene, the CD25 gene, or the TOX gene can be deleted from the genome using CRISPR or TALEN. Differentiation of the T cells into T regulatory cells can be attenuated with an inhibitor or by genetic modification. Differentiation of the T cells into T regulatory cells can be attenuated through inhibition of PI3Kδ, Foxp3, CD25, or Ezh2. PI3Kδ, Foxp3, CD25, or Ezh2 can be inhibited with a PI3Kδ inhibitor, a Foxp3 inhibitor, a CD25 inhibitor, or a Ezh2 inhibitor or by genetic modification. The genetic modification can comprise deletion of the PI3Kδ gene, the Foxp3 gene, the CD25 gene, or the Ezh2 gene. Differentiation of the T cells into T regulatory cells or the function of regulatory T cells can be attenuated with a small molecule, a small interfering RNA (siRNA), or short hairpin RNA (shRNA) against BTK, ITK, PI3Kδ, Helios, Blimp1, SOCS1, Foxp3, GATA3, IL-10, STAT3, TGF-beta Receptor II, LAG-3, PD-1, TNF-alpha, Ezh2, CD25, or TOX.

The isolated immune cells can be obtained from a donor subject. The donor subject can be mismatched to a recipient subject for at least one human leukocyte antigen (HLA) Class II allele in the donor versus recipient (graft-versus-host) direction relative to the recipient subject. The donor CD4+ T cells can be stimulated in vivo or ex vivo by an antigen present in a recipient subject, and the donor subject comprises at least one HLA Class II allele match relative to the recipient.

The immune cells obtained from the donor subject (i) can be mismatched to a recipient subject for at least one HLA Class II allele mismatch in the donor versus recipient (graft-versus-host) direction relative to the recipient subject and (ii) the donor CD4+ T cells can be stimulated in vivo or ex vivo by an antigen present in a recipient subject, and the donor subject can comprise at least one human leukocyte HLA Class II allele match relative to the recipient. The HLA Class II match is an HLA-DRB1 allele, an HLA-DQB1 allele, or an HLA-DPB1 allele.

The isolated immune cells can be autologous immune cells.

### DETAILED DESCRIPTION OF THE INVENTION

The disclosure relates to compositions suitable for adoptive cell therapy that comprises immune cells that are modified for enhanced in vivo anti-tumor activity. In particular, the compositions suitable for adoptive cell therapy are enriched for CD4+ Th1 cells and optionally depleted of CD8+ T cells.

Without wishing to be bound by any particular theory, it is believed that the adoptive cell therapy composition can lead to greater immune recruitment, proliferation and effector function in the tumor microenvironment. When the adoptive cell therapy is part of an allogeneic transplant, the risk of sustained engraftment and graft-versus-host disease is reduced. This is expected to help limit well-known challenges and adverse events of adoptive cell therapy such as cytokine release syndrome, neurotoxicity, on-target but off tumor toxicity, and immune exhaustion. In particular, the inventors believe that the adoptive cell therapy compositions disclosed herein can provide a source of CD4+ T cells that can provide signals to decrease immune suppression, and/or increase immune system activation, and/or reverse the exhaustion of CD8+ T cells upon transfusion and revive the endogenous anti-tumor response.

This disclosure relates to the use of an adoptive cell therapy composition to increase the efficacy of adoptive cell therapies by, for example, increasing the ability of the therapeutic cells to proliferate and/or deliver effector functions at the desired site of activity. This may, for example, overcome the immunosuppressive effect of the tumor microenvironment, increasing the effectiveness of adoptive cell therapies in solid tumors.

Without committing to a particular mechanism, the modifications may increase the frequency of type 1, or Th1 CD4+ T cells, which secrete interferon gamma (IFNg) and provide optimal help for recipient CD8+ T cells, or render CD4+ T cells more resistant to exhaustion or suppression of activity. The adoptive cell therapy composition can provide a source of CD4+ T cell help to reverse exhaustion of a subject's T cells. In addition to providing help to revive exhausted CD8+ T cells, Th1 cells can re-program the tumor microenvironment to become immunostimulatory. When the T cells are from a donor, Th1 cells can reverse exhaustion of CD8+ T cells while limiting the toxicities of graft versus host disease.

As disclosed above, the immune cells in present in adoptive cell therapy composition disclosed herein are modified to enrich for CD4+ Th1 cells. The immune cells can be modified to suppress Bruton tyrosine kinase (BTK), Interleukin-2-inducible T cell kinase (ITK), phosphatidyl inositol 3-kinase delta isoform (PI3Kδ), helios, blimp1, SOCS1, GATA3, IL-10, STAT3, TOX, CD25, foxp3, Ezh2, TGF-beta Receptor II, LAG-3, PD-1, TNF-alpha, or combinations thereof. Without wishing to be bound by theory or mechanism, the inventors believe that suppression of BTK, ITK, PI3Kδ, helios, blimp1, SOCS1, GATA3, IL-10, STAT3, TOX, CD25, foxp3, Ezh2, TGF-beta Receptor II, LAG-3, PD-1, TNF-alpha, or combinations thereof may promote naïve CD4+ T cells to differentiate to a state, such as type 1 (Th1) CD4+ T cells, that is favorable for helping effector cells of anti-tumor or anti-viral immunity, or prevent post-naïve CD4+ T cells from converting to cells with suboptimal helper activity for anti-tumor or anti-viral immunity. For example, a portion of the T cells may be preferentially differentiated to a CD4+ T cell sub-type (e.g., Th1). Suppression of BTK, ITK, PI3Kδ, helios, blimp1, SOCS1, GATA3, IL-10, STAT3, TOX, CD25, foxp3, Ezh2, TGF-beta Receptor II, LAG-3, PD-1, TNF-alpha, or combinations thereof may also prevent naïve CD4+ T cells from differentiating to states, such as Th2, Th17, or regulatory T cell, that are suboptimal for promoting anti-tumor or anti-viral immunity, or may prevent CD4+ T cells from becoming exhausted or suppressed by other cells from mediating anti-tumor or anti-viral activity.

The adoptive cell therapy compositions disclosed herein preferably provides therapeutic benefit for cancer. For example, the adoptive cell therapy composition can comprise immune cells that recognize and provide effector function to kill tumor cells. In some embodiments, the adoptive cell therapy composition further comprises immune cells that have been genetically modified, such as to provide specificity for target antigens, for example target antigens that are preferentially expressed by tumors. In some embodiments, such immune cells express chimeric antigen receptors (CAR) or modified T cell receptors (TCR). In some embodiments, the adoptive cell therapy comprises immune cells that are capable of killing tumor cells. In some embodiments, the adoptive cell therapy composition comprises tumor infiltrating lymphocytes (TILs).

Certain illustrative and preferred embodiments are described in further detail herein. The embodiments within the specification should not be construed to limit the scope of the disclosure.

### A. Adoptive Cell Therapy Compositions and Immune Cells

The adoptive cell therapy composition disclosed herein comprises immune cells that are enriched for CD4+ Th1 cells. The composition can additionally comprise any desired immune cells, such as T cells, B cells, NK cells and the like and any combination of immune cells. For example, the adoptive cell therapy can be a substantially homogenous population of T cells, such as CAR-T cells. In other examples, the cell therapy can contain CD4+ Th1 T cells and one or more cell types, such as primary cells that have been expanded ex-vitro and are administered to the subject in need thereof. A cell therapy for cancer can include, for example, dendritic cells, B cells, T cells (e.g., CD3+ T cells, CD4+ T cells, CD8+ T cells, NKT cells, alpha beta T cells, gamma delta T cells, etc.), NK cells, and/or macrophages.

The adoptive cell therapy composition disclosed herein can comprise immune cells that are engineered. For example, an immune cell may be engineered to express an antigen binding protein such as a Chimeric Antigen Receptor (CAR) or a T cell receptor (TCR) subunit. An “antigen binding protein” (ABP) is a protein comprising one or more antigen-binding domains that specifically bind to an antigen or epitope. Immune cells such as T cells may be engineered to express a CAR or TCR in order to make the cell specific for an antigen of interest, such as a tumor antigen. Preferably, an antigen binding protein expressed by an immune cell directs the immune cell and its immune effector functions to cells that express a desired antigen. An immune cell may be engineered using any method, such as via viral vectors, CRISPR, TALEN, or meganucleases. Immune cells can be engineered or genetically modified using any suitable approach, in vitro or in vivo. For in vitro engineering, cells are typically cultured and genetically modified using any suitable method, such as viral transduction. Engineered cells can then be selected and if desired expanded and administered to a subject as adoptive cell therapy. For in vivo engineering, typically an engineered genetic construct is administered to the patient to transduce immune cells. A number of suitable approaches can be used such as, for example, viral vectors that infect immune cells and carry a desired transgene, or other suitable nucleic acid delivery technology. Alternatively, an immune cell may not have been engineered. For example, TIL therapy often does not involve engineering the therapeutic cells because they are capable of killing tumor cells. However, TILs may be selected and expanded, for example ex vivo, to produce a population of cells with specificity for a tumor antigen on the subject's tumor.

In some embodiments, the adoptive cell therapy composition disclosed herein can comprise one or more immune cells that expresses an antigen binding protein that directs the immune cell and its immune effector functions to cells that express a desired antigen. In some embodiments, an antigen binding protein is a Chimeric Antigen Receptor (CAR). In some embodiments, an antigen binding protein is a T cell Receptor (TCR) or TCR subunit, such as a TCR beta chain or TCR alpha chain. In some embodiments, an antigen binding protein is a tumor infiltrating lymphocyte (TIL).

The antigen binding protein can be expressed on a T cell, such as an alpha beta T cell, a gamma delta T cell, a CD8+ T cell, or a CD4+ T cell. In embodiments, the antigen binding protein is preferably expressed on an enriched CD4+ T cell.

In some embodiments, the antigen binding protein specifically binds to a tumor antigen. The antigen binding protein can be CD19, CD123, CD22, CD30, CD171, CS-1, CLL-1 (CLECL1), CD33, EGFRvIII, GD2, GD3, BCMA, Tn Ag, PSMA, ROR1, FLT3, TAG72, CD38, CD44v6, CEA, EPCAM, B7H3, KIT, IL-13Ra2, Mesothelin, IL-11Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, FAP, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, TSHR, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, legumain, HPV E6, E7, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, fibronectin EDB (EDB-FN), 5T4 oncofetal antigen, and IGLL1.

a. Chimeric Antigen Receptors

Chimeric antigen receptors (CARs) are genetically engineered receptors. Immune cells, such as T cells, can be engineered to express these receptors in order to enhance their ability to target an antigen, such as a tumor antigen. In some embodiments, a CAR comprises an antigen binding domain that specifically binds to a target antigen, a hinge domain, a transmembrane domain, a costimulatory domain, and a primary signaling domain. In some embodiments, a cell contains multiple CARs targeting different antigens, such as CD19 and CD20.

CAR may be engineered to bind to a target antigen (such as a cell surface antigen) by incorporating an antigen binding domain specific for the target antigen. In some embodiments, the antigen binding domain is an antibody or a fragment thereof. In some embodiments, an antigen binding domain is a single chain antibody fragment (scFv) comprising an antibody fragment comprising the variable region of a light chain and an antibody fragment comprising the variable region of a heavy chain that are linked and expressed as a single chain polypeptide, and retain the specificity of the antibody from which they are derived. Typically, the heavy chain and light chain are connected by a short polypeptide linker. Unless specified otherwise, the VH and VL may be in either order. In some embodiments, an antigen binding domain is specific for a tumor antigen described herein.

In some embodiments, a hinge domain may be located between an extracellular domain (comprising the antigen binding domain) and a transmembrane domain of a CAR, or between a cytoplasmic domain and a transmembrane domain of the CAR. A hinge domain can be any oligopeptide or polypeptide that functions to link the transmembrane domain to the extracellular domain and/or the cytoplasmic domain in the polypeptide chain. A hinge domain may function to provide flexibility to the CAR, or domains thereof, or to prevent steric hindrance of the CAR, or domains thereof.

As used herein, a “transmembrane domain” refers to any protein structure that is thermodynamically stable in a cell membrane, preferably a eukaryotic cell membrane. In some embodiments, a transmembrane domain is a hydrophobic alpha helix that spans the membrane. In some embodiments, the transmembrane domain is a CD8 or CD28 transmembrane domain.

In some embodiments, a CAR comprises at least one intracellular costimulatory domain selected from the group CD28, 4-1BB, ICOS, CD27, and OX40.

CARs typically contain the intracellular signaling domain of CD3zeta. CD3zeta is the cytoplasmic signaling domain of the T cell receptor complex. CD3z contains 3 immunoreceptor tyrosine-based activation motif (ITAM)s, which transmit an activation signal to the T cell after the T cell is engaged with a cognate antigen. In many cases, CD3zeta provides a primary T cell activation signal but not a fully competent activation signal, which requires a co-stimulatory signaling.

b. T Cell Receptors

A TCR is composed of two different and separate protein chains, namely the TCR alpha (a) and the TCR beta (b) chain. The TCR a chain comprises variable (V), joining (J) and constant (C) regions. The TCR b chain comprises variable (V), diversity (D), joining (J) and constant (C) regions. The rearranged V(D)J regions of both the TCR a and the TCR b chain contain hypervariable regions (CDR, complementarity determining regions), among which the CDR3 region determines the specific epitope recognition. At the C-terminal region both the TCR a chain and TCR b chain contain a hydrophobic transmembrane domain and end in a short cytoplasmic tail. Typically, the TCR is a heterodimer of one a chain and one b chain. This heterodimer can bind to MHC molecules presenting a peptide.

In some embodiments, an antigen binding protein is a TCR subunit, such as an alpha, beta, gamma, or delta chain. In some embodiments, an immune cell is engineered to express a single TCR subunit that is incorporated into endogenous TCRs. In some embodiments, an immune cell is engineered to express multiple TCR subunits.

c. Tumor Infiltrating Lymphocytes

The terms “tumor infiltrating lymphocytes” or “TILs” refer to a population of cells originally obtained as white blood cells that have left the bloodstream of a subject and migrated into a tumor. TILs typically include CD8+ and CD4+ T cells (e.g., CD8+ cytotoxic T cells, CD4+ Th1 T cells and/or CD4+ Th17 T cells). TIL cell preparations can also include other cell types, such as natural killer cells, dendritic cells and M1 macrophages. TILs include both primary and secondary TILs. “Primary TILs” are those that are obtained from subject tissue samples, and “secondary TILs” are any TIL cell populations that have been expanded or proliferated as discussed herein, including, but not limited to bulk TILs and expanded TILs. TIL cell populations can include genetically modified TILs.

In some embodiments, a TIL has not been genetically engineered. In some embodiments, a TIL has been modified to express an antigen binding protein or a inducible cytokine prodrug. In some embodiments, a subject in need thereof is treated with autologous TILs.

TILs and production of TILs are described, for example, in International Patent Application PCT/US2018/064135. In some embodiments, TILs are selected based on their antigen specificity.

d. Autologous and Allogeneic Compositions

The adoptive cell therapy composition disclosed herein can include immune cells that are obtained from the subject (i.e., autologous) or obtained from a donor (i.e., allogeneic). When the T cells are obtained from a donor, the donor can comprise at least one HLA class II allele mismatch relative to the recipient in the donor versus the recipient (graft-versus-host, or GVH) direction. The HLA class II allele mismatch can be at HLA-DRB1, HLA-DQB1, or HLA-DPB1.

Donors that have not been vaccinated against one or more tumor-specific antigens, including neoantigens, generally have a low frequency of tumor-specific CD4+ T cells. When using a donor whose immune system has not been vaccinated against one or more tumor-specific antigens, no HLA Class II matching is required between the donor and the recipient because the ability to revive endogenous anti-tumor immunity is based on the activity of alloreactive CD4+ T cells.

Some degree of HLA Class II allele matching is required when vaccinating the donor against tumor-specific antigens or when expanding tumor-specific CD4+ T cells ex vivo since the expanded tumor-specific CD4+ T cells are restricted to donor HLA Class II molecules and are predicted to be ineffective at delivering help in the recipient unless the recipient expresses at least one HLA Class II molecule that is shared by the donor. Among HLA Class II molecules, the preferred molecules for sharing are the high expression molecules HLA-DRB1>HLA-DPB1>HLA-DQB1>>HLADRB3,4,5=HLA-DQA1.

The donor can be partially or completely mismatched at HLA class II alleles in the donor anti-recipient (GVH) direction, for example HLA-DRB1, HLA-DQB1, and HLA-DPB1. The donor can be partially or completely mismatched at HLA class II alleles, for example HLA-DRB1, HLA-DQB1, and HLA-DPB1 and completely matched for Class I alleles. The donor can be completely mismatched with unshared HLAs of first-degree relatives of the recipient who are potential donors for allogeneic stem cell transplantation.

Donor leukocytes may be stimulated in vivo or ex vivo to increase the frequency, compared to the unstimulated leukocytes, of CD4+ T cells that proliferate and/or secrete IFNγ in response to a tumor or viral antigen. The stimulation may consist of deliberate in vivo vaccination of the donor against a tumor antigen or a viral antigen. Alternatively, or in addition, donor leukocytes containing CD4+ T cells can be stimulated ex vivo using antigen-presenting cells (APCs), such as dendritic cells, pulsed with a tumor or viral antigen in the presence or absence of CD4+ T cell-polarizing cytokines. The tumor antigen or viral antigen can be present in the recipient. In instances in which the donor is immunized or donor cells are stimulated ex vivo with antigen pulsed-APCs, the donor must comprise at least one HLA Class II allele match relative to the recipient. The HLA class II allele match can be at HLA-DRB1, HLA-DQB1, or HLA-DPB1. In embodiments, the immunized donor can have at least one HLA class II allele mismatch relative to the recipient in the donor versus the recipient (graft-versus-host) direction and at least one HLA Class II allele match relative to the recipient. When the donor leukocytes have not been stimulated to increase the frequency of tumor- or virus-specific CD4+ T cells, the donor HLA Class II molecules HLA-DRB1, HLA-DQB1, and HLA-DPB1 may be fully mismatched to the recipient in the donor anti-recipient (GVH) direction.

A donor sample can be obtained from a cord blood bank. When the donor sample is obtained from a cord blood bank, a desirable sample may include non-frequent and/or rare HLA alleles as a subject is less likely to contain serum antibodies to non-frequent and/or rare HLA allele types. Exemplary rare alleles include, but are not limited to, A*24:41, B*07:02:28, B*35:03:03, B*39:40N, DRB1*13:23, DRB1*14:111, B*44:16 and DRB1*01:31, C*06:49N, B*37:03N, A*24:312N, and A*30:76N.

In embodiments, the recipient may not have detectable antibodies reactive against HLA of the donor. Detectable antibodies can be determined using conventional methods known to those of skill in the art. For example, the recipient may not have antibodies against donor HLA molecules that are detectable by complement-dependent cytotoxicity, in flow cytometric cross-match assays as a positive result is undesirable, or mean fluorescence intensity (MFI) of 3000 or greater in a solid phase immunoassay is unacceptable.

In embodiments, the CD4+ T cells present in the adoptive cell therapy compositions disclosed herein are not activated ex vivo.

The leukocytes present in the adoptive cell therapy composition can be depleted of CD8+ T cells. CD8+ T cells can be depleted using any known methods. For example, magnetic bead cell sorters or flow cytometry may be used to deplete the CD8+ T cells. Reducing CD8+ T cells can involve using an anti-CD8+ antibody associated with a magnetic particle or an anti-CD8+ antibody plus complement.

The leukocytes can be depleted of CD8+ T cells by about 1 fold, about 2 fold, about 3 fold, about 4 fold, about 5 fold, about 6 fold, about 7 fold, about 8 fold, about 9 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 200 fold, about 300 fold, about 400 fold, about 500 fold, about 600 fold, about 700 fold, about 800 fold, about 900 fold, about 1,000 fold or greater relative to undepleted leukocytes.

### B. Modified Adoptive Cell Therapy Compositions

The immune cells of the adoptive cell therapy compositions disclosed herein can be modified to suppress the activity of BTK, ITK, or PI3Kδ, helios, blimp1, SOCS1, GATA3, IL-10, STAT3, TOX, CD25, foxp3, Ezh2, TGF-beta Receptor II, LAG-3, PD-1, TNF-alpha, or combinations thereof. Without wishing to be bound by theory or mechanism, the inventors believe that suppression of BTK, ITK, PI3Kδ, helios, blimp1, SOCS1, GATA3, IL-10, STAT3, TOX, CD25, foxp3, Ezh2, TGF-beta Receptor II, LAG-3, PD-1, TNF-alpha, or combinations thereof may promote naïve CD4+ T cells to differentiate to a state, such as type 1 (Th1) CD4+ T cells, that is favorable for helping effector cells of anti-tumor or anti-viral immunity, or prevent post-naïve CD4+ T cells from converting to cells with suboptimal helper activity for anti-tumor or anti-viral immunity. For example, a portion of the T cells may be preferentially differentiated to a CD4+ T cell sub-type, such as Th1. Alternatively, differentiation of T cells into another CD4+ T cell subtype (e.g., Th2 or Treg) can be suppressed. Without wishing to be bound by theory or mechanism the inventors also believe that suppression of BTK, ITK, PI3Kδ, helios, blimp1, SOCS1, GATA3, IL-10, STAT3, TOX, CD25, foxp3, Ezh2, TGF-beta Receptor II, LAG-3, PD-1, TNF-alpha, or combinations thereof in leukocytes (e.g., donor leukocytes) augments their efficacy and may reduce the toxicity of non-engrafting allogenic donor lymphocyte infusions.

CD4+ T cells are T lymphocytes that express T cell receptors recognizing peptide antigens presented in the context of Class II major histocompatibility complex (MHC II) molecules. Tay et. al. (2021), Cancer Gene Therapy, 28:5-17. CD4+ T cells can differentiate into one of several diverse functional subtypes in response to context-dependent signals, which in turn allows them to provide ‘help’ to appropriate effector immune cells in their primary role as central coordinators of the immune response. CD4+ T cells primarily mediate anti-tumor immunity by providing help for CD8+ T cells and antibody responses, by inducing tumoricidal capacity of macrophages, by secretion of effector cytokines such as IFNγ and tumor necrosis factor-α (TNFα), and, under specific contexts, via direct cytotoxicity against tumor cells.

CD4+ T cells can differentiate into Th1 cells that express IFNγ and TNFα, Th2 cells that express IL-4, IL-5, and IL-13; Th9 cells that express IL-9 and IL-21; Th17 cells that expresses IL-17; TFH cells that express IL-6 and IL-21; and Treg cells that express TGFβ and IL-10. Tay et. al. (2021), Cancer Gene Therapy, 28:5-17.

As disclosed above, the immune cells of the adoptive cell therapy compositions disclosed herein can be modified to inhibit BTK, ITK, or PI3Kδ, helios, blimp1, SOCS1, GATA3, IL-10, STAT3, TOX, CD25, foxp3, Ezh2, TGF-beta Receptor II, LAG-3, PD-1, TNF-alpha, or combinations thereof. The BTK pathway can be inhibited alone or with ITK, or PI3Kδ, or combinations thereof. The ITK pathway can be inhibited alone or with BTK, or PI3Kδ, or combinations thereof. The PI3Kδ pathway can be inhibited alone or with BTK, or ITK, or combinations thereof. In some instances, each of BTK, ITK, or PI3Kδ pathways are inhibited. Helios can be inhibited alone or in combination with BTK, ITK, or PI3Kδ, Blimp1, SOCS1, Foxp3, GATA3, IL-10, STAT3, CD25, Ezh2, TGF-beta Receptor II, LAG-3, PD-1, TNF-alpha, or TOX. Blimp1 can be inhibited alone or in combination with BTK, ITK, PI3Kδ, Helios, Foxp3, GATA3, IL-10, STAT3, CD25, Ezh2, SOCS1, TGF-beta Receptor II, LAG-3, PD-1, TNF-alpha or TOX. SOCS1 can be inhibited alone or in combination with BTK, ITK, PI3Kδ, Blimp1, Helios, Foxp3, GATA3, IL-10, STAT3, CD25, Ezh2, TGF-beta Receptor II, LAG-3, PD-1, TNF-alpha, or TOX. TGF-beta Receptor II can be inhibited alone or in combination with BTK, ITK, PI3Kδ, Blimp1, Helios, Foxp3, GATA3, IL-10, STAT3, CD25, Ezh2, SOCS1, LAG-3, PD-1, TNF-alpha, or TOX. LAG-3 can be inhibited alone or in combination with BTK, ITK, PI3Kδ, Blimp1, Helios, Foxp3, GATA3, IL-10, STAT3, CD25, Ezh2, TGF-beta Receptor II, SOCS1, PD-1, TNF-alpha, or TOX. PD-1 can be inhibited alone or in combination with BTK, ITK, PI3Kδ, Blimp1, Helios, Foxp3, GATA3, IL-10, STAT3, CD25, Ezh2, TGF-beta Receptor II, LAG-3, SOCS1, TNF-alpha, or TOX. TNF-alpha can be inhibited alone or in combination with BTK, ITK, PI3Kδ, Blimp1, Helios, Foxp3, GATA3, IL-10, STAT3, CD25, Ezh2, TGF-beta Receptor II, LAG-3, PD-1, SOCS1, or TOX. Foxp3 can be inhibited alone or in combination with BTK, ITK, PI3Kδ, Helios, Blimp1, SOCS1, Foxp3, GATA3, IL-10, STAT3, CD25, TGF-beta Receptor II, LAG-3, PD-1, TNF-alpha, Ezh2, or TOX. GATA3 can be inhibited alone or with BTK, ITK, or PI3Kδ, Helios, Blimp1, SOCS1, GATA3, IL-10, STAT3, CD25, TGF-beta Receptor II, LAG-3, PD-1, TNF-alpha, Ezh2, or TOX. GATA3 can be inhibited alone or with BTK, ITK, or PI3Kδ, Helios, Blimp1, SOCS1, Foxp3, IL-10, STAT3, CD25, Ezh2, TGF-beta Receptor II, LAG-3, PD-1, TNF-alpha, or TOX. IL-10 can be inhibited alone or with BTK, ITK, PI3Kδ, Helios, Blimp1, SOCS1, Foxp3, GATA3, TGF-beta Receptor II, LAG-3, PD-1, TNF-alpha, STAT3, or TOX. STAT3 can be inhibited alone or in combination with BTK, ITK, or PI3Kδ, Helios, Blimp1, SOCS1, Foxp3, GATA3, IL-10, CD25, TGF-beta Receptor II, LAG-3, PD-1, TNF-alpha, Ezh2, or TOX. TOX can be inhibited alone or with BTK, ITK, PI3Kδ, Helios, Blimp1, SOCS1, Foxp3, GATA3, IL-10, CD25, TGF-beta Receptor II, LAG-3, PD-1, TNF-alpha, Ezh2, or STAT3. CD25 can be inhibited alone or with BTK, ITK, or PI3Kδ, Helios, Blimp1, SOCS1, Foxp3, IL-10, STAT3, GATA3, Ezh2, TGF-beta Receptor II, LAG-3, PD-1, TNF-alpha, or TOX. IL-10 can be inhibited alone or with BTK, ITK, or PI3Kδ, Helios, Blimp1, SOCS1, Foxp3, GATA3, STAT3, TGF-beta Receptor II, LAG-3, PD-1, TNF-alpha, or TOX. Ezh2 can be inhibited alone or with BTK, ITK, PI3Kδ, Helios, Blimp1, SOCS1, Foxp3, IL-10, STAT3, CD25, GATA3, TGF-beta Receptor II, LAG-3, PD-1, TNF-alpha, or TOX. IL-10 can be inhibited alone or with BTK, ITK, or PI3Kδ, Helios, Blimp1, SOCS1, Foxp3, GATA3, STAT3, TGF-beta Receptor II, LAG-3, PD-1, TNF-alpha, or TOX.

The adoptive cell therapy compositions can be modified to inhibit BTK. The adoptive cell therapy compositions can be modified to inhibit ITK. The adoptive cell therapy compositions can be modified to inhibit PI3Kδ. The adoptive cell therapy compositions can be modified to inhibit BTK and PI3Kδ. The adoptive cell therapy compositions can be modified to inhibit BTK and ITK. The adoptive cell therapy compositions can be modified to inhibit ITK and PI3Kδ. The adoptive cell therapy compositions can be modified to inhibit BTK, ITK, and PI3Kδ. The adoptive cell therapy compositions can be modified to inhibit helios. The adoptive cell therapy compositions can be modified to inhibit blimp1. The adoptive cell therapy compositions can be modified to inhibit SOCS1. The adoptive cell therapy compositions can be modified to inhibit TGF-beta Receptor II. The adoptive cell therapy compositions can be modified to inhibit LAG-3. The adoptive cell therapy compositions can be modified to inhibit PD-1. The adoptive cell therapy compositions can be modified to inhibit TNF-alpha. The adoptive cell therapy compositions can be modified to inhibit GATA3. The adoptive cell therapy compositions can be modified to inhibit IL-10. The adoptive cell therapy compositions can be modified to inhibit STAT3. The adoptive cell therapy compositions can be modified to inhibit TOX. The adoptive cell therapy compositions can be modified to inhibit CD25. The adoptive cell therapy compositions can be modified to inhibit foxp3. The adoptive cell therapy compositions can be modified to inhibit Ezh2.

The adoptive cell therapy compositions can be modified to attenuate differentiation of CD4+ T cells into T regulatory (Treg) cells or inhibit CD4+ Treg function. Suppression of PI3Kδ can attenuate differentiation into Treg cells or Treg function. Suppression of Foxp3 can attenuate differentiation into Treg cells. Suppression of CD25 can attenuate differentiation into Treg cells. Suppression of Ezh2 can attenuate differentiation into Treg cells.

Differentiation of the T cells into Treg cells can be attenuated by modifying the isolated leukocytes to express a dominant negative transforming growth factor-beta RII receptor. Liu et al., Nature, 2020, 587(7832):115-120.

BTK, ITK, or PI3Kδ, helios, blimp1, SOCS1, GATA3, IL-10, STAT3, TOX, CD25, TGF-beta Receptor II, LAG-3, PD-1, TNF-alpha, foxp3, Ezh2, or combinations thereof can be suppressed (e.g., inhibited) with a pharmacological agent. Alternatively, BTK, ITK, or PI3Kδ, helios, blimp1, SOCS1, GATA3, IL-10, STAT3, TOX, CD25, TGF-beta Receptor II, LAG-3, PD-1, TNF-alpha, foxp3, Ezh2, or combinations thereof can be inhibited by genetic modification.

Th1-mediated events can contribute to toxicities of immunotherapies including cytokine release syndrome (Imus et al., Biol Blood Marrow Transplant, (2019), 25(12):2431-2437) or liver toxicity (Guan et al., Cell Death Dis., (2021), 12(5):431.) via the effects on innate immune cells, such as macrophages and neutrophils. Without wishing to be bound by theory or mechanism, inhibition of BTK, ITK, helios, blimp1, SOCS1, GATA3, IL-10, STAT3, TOX, CD25, foxp3, Ezh2, TGF-beta Receptor II, LAG-3, PD-1, TNF-alpha, or combinations thereof may also reduce or prevent cytokine release syndrome. Suppression of BTK, ITK, helios, blimp1, SOCS1, GATA3, IL-10, STAT3, TOX, CD25, foxp3, Ezh2, TGF-beta Receptor II, LAG-3, PD-1, TNF-alpha, or combinations thereof my reduce or prevent cytokine release syndrome through inhibition of myeloid cell activation.

CD4+ T cells in the leukocytes can be measured using conventional methodologies known by those skilled in the art, for example, flow cytometry. The expression level of cytokines expressed by the CD4+ T cells can be measured using conventional methodologies by those skilled in the art, for example ELISA or intracellular cytokine staining followed by cell surface staining and flow cytometry.

i. BTK

BTK is a member of the Tec family of non-receptor tyrosine kinases, which consists of a PH domain, a TH domain, an SH3 domain, an SH2 domain, and a catalytic domain. BTK is involved in the signaling of multiple receptors including growth factor receptors, cytokine receptors, G-protein coupled receptors, antigen receptors and integrins. BTK in turn activates many of the major downstream signaling pathways that control cell migration, adhesion, survival and proliferation.

BTK can be suppressed (i.e., inhibited) with a pharmacological agent or by genetic modification. The pharmacological agent could be an inhibitor of BTK. Suitable inhibitors of BTK include, but are not limited to, acalabrutinib, zanubrutinib, tirabrutinib, evobrutinib, tolebrutinib, rilzabrutinib, remibrutinib, branebrutinib, orelabrutinib, BIIB091, AC0058, PRN473LFM-A13, dasatinib, GD-4059, or AVL-292. The inhibitor can be a small molecule, a small interfering RNA (siRNA), or short hairpin RNA (shRNA). In embodiments, the BTK inhibitor used herein is not ibrutinib. The BTK inhibitor can have a half-maximal inhibitory concentration of less than about 1000 nM, about 900 nM, about 800 mM, about 700 nM, about 600 nM, about 500 nM, about 400 nM, about 300 nM, about 200 nM, about 100 nM or less.

BTK can be suppressed by deleting, or “knocking out” the BTK gene from the genome. Techniques for knocking out genes are known by those skilled in the art. Gene knock-out methods in the art include, but are not limited to, gene silencing, conditional knockout, homologous recombination, gene editing, and knockout by mutation. Gene silencing can be achieved using, for example, RNA interference, siRNA or shRNA. Conditional knockout methods can be used to inactivate the BTK gene. A loss of function mutation can help to suppress gene function by creating a mutation in the BTK gene. Gene editing techniques that can be employed to suppress BTK include, but are not limited to, zinc-finger nucleases (ZFN), transcription activator-like effector nucleases (TALEN), meganucleases, and CRISPR-based systems (e.g., CRISPR-Cas9). Commercially available kits can be employed to suppress BTK. A mutation can be made in one or more of the protein domains. For example, a mutation can be made in the pleckstrin homology domain, the proline-rich TEC homology (TH) domain, or the SRC homology domains (SH2 or SH3).

Suppression of BTK can decrease the number or frequency of Th2-polarized T cells in the leukocytes. Suppression of BTK can increase the number or frequency of Th1-polarized T cells in the leukocytes. Suppression of BTK can promote differentiation of T cells to Th1. Suppression of BTK can decrease IL-10, IL-4, or IL-13 expression in the leukocytes. Suppression of BTK can increase IFN-γ expression in the leukocytes. Suppression of BTK can increase IL-12 expression in the leukocytes.

Suppression of BTK can increase the population of Th1 cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where BTK is not suppressed. Suppression of BTK can decrease the population of Th2 by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where BTK is not suppressed.

Suppression of BTK can increase the expression of one or more Th1 cell-related markers. Suppression of BTK can increase the expression of one or more Th1 cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where BTK is not suppressed. The one or more Th1 related markers can include CCR1, CD4, CD26, CD94, CD119, CD183, CD195, CD212, GM-CSF, Granzyme B, IFN-α, IFN-γ, IL-2, IL-12, IL-15, IL-18R, IL-23, IL-27, IL-27R, Lymphotoxin, perforin, t-bet, Tim-3, TNF-α, TRANCE, sCD40L, or any combination thereof. In particular, the one or more Th1 related markers can include IFN-γ, IL-2, IL-12 or any combination thereof. For example, suppression of BTK can increase expression of IFN-γ. For example, suppression of BTK can increase IL-2. For example, suppression of BTK can increase expression of IL-12.

Suppression of BTK can decrease the expression of one or more Th2 cell related markers. Suppression of BTK can decrease the expression of one or more Th2 cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where BTK is not suppressed. The one or more Th2 related markers can include CCR3, CCR4, CCR7, CCR8, CD4, CD30, CD81, CD184, CD278, c-maf, CRTH2, Gata-3, GM-CSF, IFN yR, IgD, IL-1R, IL-4, IL-5, IL-6, IL-9, IL-10, IL-13, IL-15, ST2L/T1, Tim-1, or any combination thereof. In particular, the one or more Th2 related markers can include IL-4, IL-5, IL-6, IL-10, IL-13, IL-15 or any combination thereof. For example, suppression of BTK can decrease IL-4 expression. For example, suppression of BTK can decrease IL-5 expression. For example, suppression of BTK can decrease IL-6 expression. For example, suppression of BTK can decrease IL-10 expression. For example, suppression of BTK can decrease IL-13 expression. For example, suppression of BTK can decrease IL-15 expression.

Suppression of BTK can increase the ratio of Th1 T cells to Th2 T cells by about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

Suppression of BTK can decrease the ratio of Th2 T cells to Th1 T cells by about 1 fold, about 2 fold, about 3 fold, about 4 fold, about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

Cytokine release syndrome is a known complication of the treatment of hematologic malignancies with chimeric antigen receptor-modified (CAR) T cells or with T cell replete, HLA-haploidentical blood or marrow transplantation. In embodiments, inhibition of BTK can attenuate cytokine release syndrome after non-engrafting, CD8-depleted lymphocyte infusion.

Cytokine release syndrome is graded on a scale from 0 to 5. Suppression of BTK can decrease the cytokine release syndrome score to 0, 1, 2, 3, or 4.

Suppression of BTK can decrease the expression of IL-10 by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where BTK is not suppressed.

Suppression of BTK can decrease the percentage of pyroptotic leukocytes among total leukocytes by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where BTK is not suppressed.

The activity of BTK, as measured for example by phosphorylation of one of its substrates (such as 1-Phosphatidylinositol-4,5-bisphosphate phosphodiesterase gamma-2; PLC-γ2) can be suppressed (i.e., inhibited) by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater relative to basal activity.

### ITK

Interleukin-2 (IL-2) inducible T cell kinase (ITK) is a non-receptor tyrosine kinase highly expressed in T cell lineages and regulates multiple aspects of T cell development and function, mainly through its function downstream of the T cell receptor.

ITK can be suppressed (i.e., inhibited or attenuated) with a pharmacological agent or by genetic modification. The pharmacological agent could be an inhibitor of ITK. Suitable inhibitors of ITK include, but are not limited to, aminothiazole, aminobenzimidazole, indole, pyridine or prn694. The inhibitor can be a small molecule, a small interfering RNA (siRNA), or short hairpin RNA (shRNA). The ITK inhibitor can have a half-maximal inhibitory concentration of less than about 1000 nM, about 900 nM, about 800 mM, about 700 nM, about 600 nM, about 500 nM, about 400 nM, about 300 nM, about 200 nM, about 100 nM or less.

ITK can be suppressed by knocking out the ITK gene from the genome. Techniques for knocking out genes are known by those skilled in the art. Gene knock-out methods in the art include, but are not limited to, gene silencing, conditional knockout, homologous recombination, gene editing, knockout by mutation. Gene silencing can be achieved using, for example, RNA interference, siRNA or shRNA. Conditional knockout methods can be used to inactivate the ITK gene. A loss of function mutation can help to suppress gene function by creating a mutation in the ITK gene. Gene editing techniques that can be employed to suppress ITK include, but are not limited to, zinc-finger nucleases (ZFN), transcription activator-like effector nucleases (TALEN), meganucleases, and CRISPR-based systems (e.g., CRISPR-Cas9). Commercially available kits can be employed to suppress ITK. A mutation can be made in one or more of the protein domains of ITK.

Suppression of ITK can decrease the number of Th2-polarized T cells in the leukocytes. Suppression of ITK can increase the number of Th1-polarized T cells in the leukocytes. Suppression of ITK can promote differentiation of T cells to Th1. Suppression of ITK can decrease IL-10, IL-4, or IL-13 expression in the leukocytes. Suppression of ITK can increase IFN-γ expression in the leukocytes. Suppression of ITK can increase IL-12 expression in the leukocytes.

Suppression of ITK can increase the population of Th1 cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where ITK is not suppressed. Suppression of ITK can decrease the population of Th2 by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where ITK is not suppressed.

Suppression of ITK can increase the expression of one or more Th1 cell related markers. Suppression of ITK can increase the expression of one or more Th1 cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where ITK is not suppressed. The one or more Th1 related markers can include CCR1, CD4, CD26, CD94, CD1 19, CD183, CD195, CD212, GM-CSF, Granzyme B, IFN-α, IFN-γ, IL-2, IL-12, IL-15, IL-18R, IL-23, IL-27, IL-27R, Lymphotoxin, perforin, t-bet, Tim-3, TNF-α, TRANCE, sCD40L, or any combination thereof. In particular, the one or more Th1 related markers can include IFN-γ, IL-2, IL-12 or any combination thereof. For example, suppression of ITK can increase expression of IFN-γ. For example, suppression of ITK can increase IL-2. For example, suppression of ITK can increase expression of IL-12.

Suppression of ITK can decrease the expression of one or more Th2 cell related markers. Suppression of ITK can decrease the expression of one or more Th2 cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where ITK is not suppressed. The one or more Th2 related markers can include CCR3, CCR4, CCR7, CCR8, CD4, CD30, CD81, CD184, CD278, c-maf, CRTH2, Gata-3, GM-CSF, IFN yR, IgD, IL-1R, IL-4, IL-5, IL-6, IL-9, IL-10, IL-13, IL-15, ST2L/T1, Tim-1, or any combination thereof. In particular, the one or more Th2 related markers can include IL-4, IL-6, IL-10, IL-13, IL-15 or any combination thereof. For example, suppression of ITK can decrease IL-4 expression. For example, suppression of ITK can decrease IL-5 expression. For example, suppression of ITK can decrease IL-6 expression. For example, suppression of ITK can decrease IL-10 expression. For example, suppression of ITK can decrease IL-13 expression. For example, suppression of ITK can decrease IL-15 expression.

Suppression of ITK can increase the ratio of Th1 T cells to Th2 T cells by about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

Suppression of ITK can decrease the ratio of Th2 T cells to Th1 T cells by about 1 fold, about 2 fold, about 3 fold, about 4 fold, about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

The activity of ITK can be suppressed (i.e., inhibited) by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater relative to basal activity.

i. PI3Kδ

PI3Kδ can be suppressed (i.e., inhibited or attenuated) with a pharmacological agent or by genetic modification. The pharmacological agent could be an inhibitor of PI3Kδ. Suitable inhibitors of PI3Kδ include, but are not limited to, idelalisib, copanlisib, duvelisib, umbralisib, ME-4401, RP6503, perifosine, buparlisib, or dactolisib. The inhibitor can be a small molecule, a small interfering RNA (siRNA), or short hairpin RNA (shRNA). The PI3Kδ inhibitor can have a half-maximal inhibitory concentration of less than about 1000 nM, about 900 nM, about 800 mM, about 700 nM, about 600 nM, about 500 nM, about 400 nM, about 300 nM, about 200 nM, about 100 nM or less.

PI3Kδ can be suppressed by knocking out the PI3Kδ gene from the genome. Techniques for knocking out genes are known by those skilled in the art. Known gene knock-out methods in the art, include but are not limited to, gene silencing, conditional knockout, homologous recombination, gene editing, and knockout by mutation. Gene silencing can be achieved using, for example, RNA interference, siRNA or shRNA. For example, the RNA interference, siRNA or shRNA can be against CD25, foxp3, or Ezh2. Conditional knockout methods can be used to inactivate the PI3Kδ gene. A loss of function mutation can help to suppress gene function by creating a mutation in the PI3Kδ gene. Gene editing techniques that can be employed to suppress PI3Kδ include, but are not limited to, zinc-finger nucleases (ZFN), transcription activator-like effector nucleases (TALEN), meganucleases, and CRISPR-based systems (e.g., CRISPR-Cas9). Commercially available kits can be employed to suppress PI3Kδ. Alternatively, a mutation can be made in one or more of the protein domains.

Genetic modification of PI3Kδ can comprise deleting the gene for CD25, foxp3, or Ezh2.

Suppression of PI3Kδ can decrease the number or function of CD4+CD25+foxp3+ regulatory T cells (Tregs) in the leukocytes. Suppression of PI3Kδ can increase the number of Th1 polarized T cells in the leukocytes. Suppression of PI3Kδ can promote differentiation of T cells to Th1. Suppression of PI3Kδ can decrease expression of TGFβ or IL-10 in the leukocytes. Suppression of PI3Kδ can increase IFN-γ expression in the leukocytes. Suppression of PI3Kδ can increase IL-12 expression in the leukocytes.

Suppression of PI3Kδ can increase the population of Th1 cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where PI3Kδ is not suppressed. Suppression of PI3Kδ can decrease the population of Treg cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where PI3Kδ is not suppressed.

Suppression of PI3Kδ can increase the expression of one or more Th1 cell related markers. Suppression of PI3Kδ can increase the expression of one or more Th1 cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where PI3Kδ is not suppressed. The one or more Th1 related markers can include CCR1, CD4, CD26, CD94, CD1 19, CD183, CD195, CD212, GM-CSF, Granzyme B, IFN-α, IFN-γ, IL-2, IL-12, IL-15, IL-18R, IL-23, IL-27, IL-27R, Lymphotoxin, perforin, t-bet, Tim-3, TNF-α, TRANCE, sCD40L, or any combination thereof. In particular, the one or more Th1 related markers can include IFN-γ, IL-2, IL-12 or any combination thereof. For example, suppression of PI3Kδ can increase expression of IFN-γ. For example, suppression of PI3Kδ can increase IL-2. For example, suppression of PI3Kδ can increase expression of IL-12.

Suppression of PI3Kδ can decrease the expression of one or more Treg cell related markers. Suppression of PI3Kδ can decrease the expression of one or more Treg cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where Treg is not suppressed. The one or more Treg related markers can include, TGFβ or IL-10 or any combination thereof. For example, suppression of PI3Kδ can decrease TGFβ expression. For example, suppression of PI3Kδ can decrease IL-10 expression.

Suppression of PI3Kδ can increase the ratio of Th1 T cells to Treg T cells by about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

Suppression of PI3Kδ can decrease the ratio of Treg T cells to Th1 T cells by about 1 fold, about 2 fold, about 3 fold, about 4 fold, about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

The activity of PI3Kδ can be suppressed (i.e., inhibited) by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater relative to basal activity.

iii. GATA3

GATA3 can be suppressed (i.e., inhibited or attenuated) with a pharmacological agent or by genetic modification. The pharmacological agent could be an inhibitor of GATA3. The inhibitor can be a small molecule, a small interfering RNA (siRNA), or short hairpin RNA (shRNA). The GATA3 inhibitor can have a half-maximal inhibitory concentration of less than about 1000 nM, about 900 nM, about 800 mM, about 700 nM, about 600 nM, about 500 nM, about 400 nM, about 300 nM, about 200 nM, about 100 nM or less.

GATA3 can be suppressed by knocking out the GATA3 gene from the genome. Techniques for knocking out genes are known by those skilled in the art. Gene knock-out methods in the art, include but are not limited to, gene silencing, conditional knockout, homologous recombination, gene editing, and knockout by mutation. Gene silencing can be achieved using, for example, RNA interference, siRNA or shRNA. Conditional knockout methods can be used to inactivate the GATA3 gene. A loss of function mutation can help to suppress gene function by creating a mutation in the GATA3 gene. Gene editing techniques that can be employed to suppress GATA3 can include, but are not limited to, zinc-finger nucleases (ZFN), transcription activator-like effector nucleases (TALEN), meganucleases, and CRISPR-based systems (e.g., CRISPR-Cas9). Commercially available kits can be employed to suppress GATA3. A mutation can be made one or more of the protein domains.

Suppression of GATA3 can increase the number of Th1 polarized T cells in the leukocytes. Suppression of GATA3 can promote differentiation of T cells to Th1. Suppression of GATA3 can decrease IL-10, IL-4, or IL-13 expression in the leukocytes. Suppression of GATA3 can increase IFN-γ expression in the leukocytes. Suppression of GATA3 can increase IL-12 expression in the leukocytes. Suppression of GATA3 can decrease the number of Th2 polarized T cells in the leukocytes.

Suppression of GATA3 can increase the population of Th1 cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where GATA3 is not suppressed. Suppression of GATA3 can decrease the population of Th2 by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where GATA3 is not suppressed.

Suppression of GATA3 can increase the expression of one or more related Th1 cell related markers. Suppression of GATA3 can increase the expression of one or more Th1 cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where GATA3 is not suppressed. The one or more Th1 related markers can include CCR1, CD4, CD26, CD94, CD1 19, CD183, CD195, CD212, GM-CSF, Granzyme B, IFN-α, IFN-γ, IL-2, IL-12, IL-15, IL-18R, IL-23, IL-27, IL-27R, Lymphotoxin, perforin, t-bet, Tim-3, TNF-α, TRANCE, sCD40L, or any combination thereof. In particular, the one or more Th1 related markers can include IFN-γ, IL-2, IL-12 or any combination thereof. For example, suppression of GATA3 can increase expression of IFN-γ. For example, suppression of GATA3 can increase IL-2. For example, suppression of GATA3 can increase expression of IL-12.

Suppression of GATA3 can decrease the expression of one or more related Th2 cell related markers. Suppression of GATA3 can decrease the expression of one or more Th2 cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where GATA3 is not suppressed. The one or more Th2 related markers can include CCR3, CCR4, CCR7, CCR8, CD4, CD30, CD81, CD184, CD278, c-maf, CRTH2, Gata-3, GM-CSF, IFN yR, IgD, IL-1R, IL-4, IL-5, IL-6, IL-9, IL-10, IL-13, IL-15, ST2L/T1, Tim-1, or any combination thereof. In particular, the one or more Th2 related markers can include IL-4, IL-5, IL-6, IL-10, IL-13, IL-15 or any combination thereof. For example, suppression of GATA3 can decease IL-4 expression. For example, suppression of GATA3 can decrease IL-5 expression. For example, suppression of GATA3 can decrease IL-6 expression. For example, suppression of GATA3 can decrease IL-10 expression. For example, suppression of GATA3 can decrease IL-13 expression. For example, suppression of GATA3 can decrease IL-15 expression.

Suppression of GATA3 can increase the ratio of Th1 T cells to Th2 T cells by about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

Suppression of GATA3 can decrease the ratio of Th2 T cells to Th1 T cells by about 1 fold, about 2 fold, about 3 fold, about 4 fold, about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

Cytokine release syndrome is a known complication of the treatment of hematologic malignancies with chimeric antigen receptor-modified (CAR) T cells or with T cell replete, HLA-haploidentical blood or marrow transplantation. In embodiments, inhibition of GATA3 can attenuate cytokine release syndrome after non-engrafting, CD8-depleted donor lymphocyte infusion. Suppression of GATA3 can decrease cytokine release syndrome by about 5%, about 10%, about 15%, about 20%, about 25%, about 30%, about 35%, about 40%, about 45%, about 50%, about 60%, about 65%, about 70%, about 75%, about 80%, about 85%, about 90%, about 95% or greater relative to activity without suppression of GATA3.

Suppression of GATA3 can decrease the expression of IL-10 by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where GATA3 is not suppressed.

Suppression of GATA3 can decrease the expression of pyroptosis by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where GATA3 is not suppressed.

The activity of GATA3 can be suppressed (i.e., inhibited) by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater relative to basal activity.

iv. STAT3

STAT3 can be suppressed (i.e., inhibited) with a pharmacological agent or by genetic modification. The pharmacological agent could be an inhibitor of STAT3. The inhibitor can be a small molecule, a small interfering RNA (siRNA), or short hairpin RNA (shRNA). The STAT3 inhibitor can have a half-maximal inhibitory concentration of less than about 1000 nM, about 900 nM, about 800 mM, about 700 nM, about 600 nM, about 500 nM, about 400 nM, about 300 nM, about 200 nM, about 100 nM or less.

STAT3 can be suppressed by knocking out the STAT3 gene from the genome. Techniques for knocking out genes are known by those skilled in the art. Gene knock-out methods in the art, include but are not limited to, gene silencing, conditional knockout, homologous recombination, gene editing, and knockout by mutation. Gene silencing can be achieved using, for example, RNA interference, siRNA or shRNA. Conditional knockout methods can be used to inactivate the STAT3 gene. A loss of function mutation can help to suppress gene function by creating a mutation in the STAT3 gene. Gene editing techniques that can be employed to suppress STAT3 can include, but are not limited to, zinc-finger nucleases (ZFN), transcription activator-like effector nucleases (TALEN), meganucleases, and CRISPR-based systems (e.g., CRISPR-Cas9). Commercially available kits can be employed to suppress STAT3. A mutation can be made one or more of the protein domains.

Suppression of STAT3 can increase the number of Th1 polarized T cells in the leukocytes. Suppression of STAT3 can promote differentiation of T cells to Th1. Suppression of STAT3 can decrease IL-10, IL-4, or IL-13 expression in the leukocytes. Suppression of STAT3 can increase IFN-γ expression in the leukocytes. Suppression of STAT3 can increase IL-12 expression in the leukocytes. Suppression of STAT3 can decrease the number of Th17 polarized T cells or Tfh polarized T cells in the leukocytes.

Suppression of STAT3 can increase the population of Th1 cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where STAT3 is not suppressed. Suppression of STAT3 can decrease the population of Th17 cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where STAT3 is not suppressed. Suppression of STAT3 can decrease the population of Tfh cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where STAT3 is not suppressed.

Suppression of STAT3 can increase the expression of one or more related Th1 cell related markers. Suppression of STAT3 can increase the expression of one or more Th1 cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where STAT3 is not suppressed. The one or more Th1 related markers can include CCR1, CD4, CD26, CD94, CD1 19, CD183, CD195, CD212, GM-CSF, Granzyme B, IFN-α, IFN-γ, IL-2, IL-12, IL-15, IL-18R, IL-23, IL-27, IL-27R, Lymphotoxin, perforin, t-bet, Tim-3, TNF-α, TRANCE, sCD40L, or any combination thereof. In particular, the one or more Th1 related markers can include IFN-γ, IL-2, IL-12 or any combination thereof. For example, suppression of STAT3 can increase expression of IFN-γ. For example, suppression of STAT3 can increase IL-2. For example, suppression of STAT3 can increase expression of IL-12.

Suppression of STAT3 can decrease the expression of one or more Treg cell related markers. Suppression of STAT3 can decrease the expression of one or more Treg cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where Treg is not suppressed. The one or more Treg related markers can include, TGFβ or IL-10 or any combination thereof. For example, suppression of STAT3 can decrease TGFβ expression. For example, suppression of STAT3 can decrease IL-10 expression.

Suppression of STAT3 can decrease the expression of one or more Tfh cell related markers. Suppression of STAT3 can decrease the expression of one or more Tfh cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where Tfh is not suppressed. The one or more Tfh related markers can include, IL-21, IL-4, or any combination thereof. For example, suppression of STAT3 can decrease IL-21 expression. For example, suppression of STAT3 can decrease IL-4 expression.

Suppression of STAT3 can increase the ratio of Th1 T cells to Treg T cells by about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

Suppression of STAT3 can decrease the ratio of Treg T cells to Th1 T cells by about 1 fold, about 2 fold, about 3 fold, about 4 fold, about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

Suppression of STAT3 can increase the ratio of Th1 T cells to Tfh T cells by about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

Suppression of STAT3 can decrease the ratio of Tfh T cells to Th1 T cells by about 1 fold, about 2 fold, about 3 fold, about 4 fold, about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

The activity of STAT3 can be suppressed (i.e., inhibited) by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater relative to basal activity.

v. Foxp3

Foxp3 can be suppressed (i.e., inhibited) with a pharmacological agent or by genetic modification. The pharmacological agent could be an inhibitor of foxp3. The inhibitor can be a small molecule, a small interfering RNA (siRNA), or short hairpin RNA (shRNA). The foxp3 inhibitor can have a half-maximal inhibitory concentration of less than about 1000 nM, about 900 nM, about 800 mM, about 700 nM, about 600 nM, about 500 nM, about 400 nM, about 300 nM, about 200 nM, about 100 nM or less.

Foxp3 can be suppressed by knocking out the FOXP3 gene from the genome. Techniques for knocking out genes are known by those skilled in the art. Gene knock-out methods in the art, include but are not limited to, gene silencing, conditional knockout, homologous recombination, gene editing, and knockout by mutation. Gene silencing can be achieved using, for example, RNA interference, siRNA or shRNA. Conditional knockout methods can be used to inactivate the FOXP3 gene. A loss of function mutation can help to suppress gene function by creating a mutation in the FOXP3 gene. Gene editing techniques that can be employed to suppress foxp3 can include, but are not limited to, zinc-finger nucleases (ZFN), transcription activator-like effector nucleases (TALEN), meganucleases, and CRISPR-based systems (e.g., CRISPR-Cas9). Commercially available kits can be employed to suppress foxp3. A mutation can be made one or more of the protein domains.

Suppression of foxp3 can increase the number of Th1 polarized T cells in the leukocytes. Suppression of foxp3 can promote differentiation of T cells to Th1. Suppression of foxp3 can decrease IL-10, IL-4, or IL-13 expression in the leukocytes. Suppression of foxp3 can increase IFN-γ expression in the leukocytes. Suppression of foxp3 can increase IL-12 expression in the leukocytes. Suppression of foxp3 can decrease the number of Treg polarized T cells in the leukocytes.

Suppression of foxp3 can increase the population of Th1 cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where foxp3 is not suppressed. Suppression of foxp3 can decrease the population of Treg cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where foxp3 is not suppressed.

Suppression of foxp3 can increase the expression of one or more related Th1 cell related markers. Suppression of foxp3 can increase the expression of one or more Th1 cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where foxp3 is not suppressed. The one or more Th1 related markers can include CCR1, CD4, CD26, CD94, CD1 19, CD183, CD195, CD212, GM-CSF, Granzyme B, IFN-α, IFN-γ, IL-2, IL-12, IL-15, IL-18R, IL-23, IL-27, IL-27R, Lymphotoxin, perforin, t-bet, Tim-3, TNF-α, TRANCE, sCD40L, or any combination thereof. In particular, the one or more Th1 related markers can include IFN-γ, IL-2, IL-12 or any combination thereof. For example, suppression of foxp3 can increase expression of IFN-γ. For example, suppression of foxp3 can increase IL-2. For example, suppression of foxp3 can increase expression of IL-12.

Suppression of foxp3 can decrease the expression of one or more related Treg cell related markers. Suppression of foxp3 can decrease the expression of one or more Treg cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where foxp3 is not suppressed. The one or more Treg related markers can include, TGFβ or IL-10 or any combination thereof. For example, suppression of foxp3 can decrease TGFβ expression. For example, suppression of foxp3 can decrease IL-10 expression.

Suppression of foxp3 can increase the ratio of Th1 T cells to Treg T cells by about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

Suppression of foxp3 can decrease the ratio of Treg T cells to Th1 T cells by about 1 fold, about 2 fold, about 3 fold, about 4 fold, about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

The activity of foxp3 can be suppressed (i.e., inhibited) by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater relative to basal activity.

vi. CD25

CD25 can be suppressed (i.e., inhibited) with a pharmacological agent or by genetic modification. The pharmacological agent could be an inhibitor of CD25. The inhibitor can be a small molecule, a small interfering RNA (siRNA), or short hairpin RNA (shRNA). The CD25 inhibitor can have a half-maximal inhibitory concentration of less than about 1000 nM, about 900 nM, about 800 mM, about 700 nM, about 600 nM, about 500 nM, about 400 nM, about 300 nM, about 200 nM, about 100 nM or less.

CD25 can be suppressed by knocking out the CD25 gene from the genome. Techniques for knocking out genes are known by those skilled in the art. Gene knock-out methods in the art, include but are not limited to, gene silencing, conditional knockout, homologous recombination, gene editing, and knockout by mutation. Gene silencing can be achieved using, for example, RNA interference, siRNA or shRNA. Conditional knockout methods can be used to inactivate the CD25 gene. A loss of function mutation can help to suppress gene function by creating a mutation in the CD25 gene. Gene editing techniques that can be employed to suppress CD25 can include, but are not limited to, zinc-finger nucleases (ZFN), transcription activator-like effector nucleases (TALEN), meganucleases, and CRISPR-based systems (e.g., CRISPR-Cas9). Commercially available kits can be employed to suppress CD25. A mutation can be made one or more of the protein domains.

Suppression of CD25 can increase the number of Th1 polarized T cells in the leukocytes. Suppression of CD25 can promote differentiation of T cells to Th1. Suppression of CD25 can decrease IL-10, IL-4, or IL-13 expression in the leukocytes. Suppression of CD25 can increase IFN-γ expression in the leukocytes. Suppression of CD25 can increase IL-12 expression in the leukocytes. Suppression of CD25 can decrease the number of Treg polarized T cells in the leukocytes.

Suppression of CD25 can increase the population of Th1 cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where CD25 is not suppressed. Suppression of CD25 can decrease the population of Treg cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where CD25 is not suppressed.

Suppression of CD25 can increase the expression of one or more related Th1 cell related markers. Suppression of CD25 can increase the expression of one or more Th1 cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where CD25 is not suppressed. The one or more Th1 related markers can include CCR1, CD4, CD26, CD94, CD1 19, CD183, CD195, CD212, GM-CSF, Granzyme B, IFN-α, IFN-γ, IL-2, IL-12, IL-15, IL-18R, IL-23, IL-27, IL-27R, Lymphotoxin, perforin, t-bet, Tim-3, TNF-α, TRANCE, sCD40L, or any combination thereof. In particular, the one or more Th1 related markers can include IFN-γ, IL-2, IL-12 or any combination thereof. For example, suppression of CD25 can increase expression of IFN-γ. For example, suppression of CD25 can increase IL-2. For example, suppression of CD25 can increase expression of IL-12.

Suppression of CD25 can decrease the expression of one or more related Treg cell related markers. Suppression of CD25 can decrease the expression of one or more Treg cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where CD25 is not suppressed. The one or more Treg related markers can include, TGFβ or IL-10 or any combination thereof. For example, suppression of CD25 can decrease TGFβ expression. For example, suppression of CD25 can decrease IL-10 expression.

Suppression of CD25 can increase the ratio of Th1 T cells to Treg T cells by about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

Suppression of CD25 can decrease the ratio of Treg T cells to Th1 T cells by about 1 fold, about 2 fold, about 3 fold, about 4 fold, about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

The activity of CD25 can be suppressed (i.e., inhibited) by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater relative to basal activity.

vii. Ezh2

Ezh2 can be suppressed (i.e., inhibited) with a pharmacological agent or by genetic modification. The pharmacological agent could be an inhibitor of Ezh2. The inhibitor can be a small molecule, a small interfering RNA (siRNA), or short hairpin RNA (shRNA). The Ezh2 inhibitor can have a half-maximal inhibitory concentration of less than about 1000 nM, about 900 nM, about 800 mM, about 700 nM, about 600 nM, about 500 nM, about 400 nM, about 300 nM, about 200 nM, about 100 nM or less.

Ezh2 can be suppressed by knocking out the Ezh2 gene from the genome. Techniques for knocking out genes are known by those skilled in the art. Gene knock-out methods in the art, include but are not limited to, gene silencing, conditional knockout, homologous recombination, gene editing, and knockout by mutation. Gene silencing can be achieved using, for example, RNA interference, siRNA or shRNA. Conditional knockout methods can be used to inactivate the Ezh2 gene. A loss of function mutation can help to suppress gene function by creating a mutation in the Ezh2 gene. Gene editing techniques that can be employed to suppress Ezh2 can include, but are not limited to, zinc-finger nucleases (ZFN), transcription activator-like effector nucleases (TALEN), meganucleases, and CRISPR-based systems (e.g., CRISPR-Cas9). Commercially available kits can be employed to suppress Ezh2. A mutation can be made one or more of the protein domains.

Suppression of Ezh2 can increase the number of Th1 polarized T cells in the leukocytes. Suppression of Ezh2 can promote differentiation of T cells to Th1. Suppression of Ezh2 can decrease IL-10, IL-4, or IL-13 expression in the leukocytes. Suppression of Ezh2 can increase IFN-γ expression in the leukocytes. Suppression of Ezh2 can increase IL-12 expression in the leukocytes. Suppression of Ezh2 can decrease the number of Treg polarized T cells in the leukocytes.

Suppression of Ezh2 can increase the population of Th1 cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where Ezh2 is not suppressed. Suppression of Ezh2 can decrease the population of Treg cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where Ezh2 is not suppressed.

Suppression of Ezh2 can increase the expression of one or more related Th1 cell related markers. Suppression of Ezh2 can increase the expression of one or more Th1 cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where Ezh2 is not suppressed. The one or more Th1 related markers can include CCR1, CD4, CD26, CD94, CD1 19, CD183, CD195, CD212, GM-CSF, Granzyme B, IFN-α, IFN-γ, IL-2, IL-12, IL-15, IL-18R, IL-23, IL-27, IL-27R, Lymphotoxin, perforin, t-bet, Tim-3, TNF-α, TRANCE, sCD40L, or any combination thereof. In particular, the one or more Th1 related markers can include IFN-γ, IL-2, IL-12 or any combination thereof. For example, suppression of Ezh2 can increase expression of IFN-γ. For example, suppression of Ezh2 can increase IL-2. For example, suppression of Ezh2 can increase expression of Ezh2.

Suppression of Ezh2 can decrease the expression of one or more related Treg cell related markers. Suppression of Ezh2 can decrease the expression of one or more Treg cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where Ezh2 is not suppressed. The one or more Treg related markers can include, TGFβ or IL-10 or any combination thereof. For example, suppression of Ezh2 can decrease TGFβ expression. For example, suppression of Ezh2 can decrease IL-10 expression.

Suppression of Ezh2 can increase the ratio of Th1 T cells to Treg T cells by about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

Suppression of Ezh2 can decrease the ratio of Treg T cells to Th1 T cells by about 1 fold, about 2 fold, about 3 fold, about 4 fold, about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

The activity of Ezh2 can be suppressed (i.e., inhibited) by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater relative to basal activity.

viii. Helios

Helios can be suppressed (i.e., inhibited) with a pharmacological agent or by genetic modification. The pharmacological agent could be an inhibitor of Helios. The inhibitor can be a small molecule, a small interfering RNA (siRNA), or short hairpin RNA (shRNA). The Helios inhibitor can have a half-maximal inhibitory concentration of less than about 1000 nM, about 900 nM, about 800 mM, about 700 nM, about 600 nM, about 500 nM, about 400 nM, about 300 nM, about 200 nM, about 100 nM or less.

Helios can be suppressed by knocking out the IKZF2 gene from the genome. Techniques for knocking out genes are known by those skilled in the art. Gene knock-out methods in the art, include but are not limited to, gene silencing, conditional knockout, homologous recombination, gene editing, and knockout by mutation. Gene silencing can be achieved using, for example, RNA interference, siRNA or shRNA. Conditional knockout methods can be used to inactivate the IKZF2 gene. A loss of function mutation can help to suppress gene function by creating a mutation in the IKZF2 gene. Gene editing techniques that can be employed to suppress Helios include, but are not limited to, zinc-finger nucleases (ZFN), transcription activator-like effector nucleases (TALEN), meganucleases, and CRISPR-based systems (e.g., CRISPR-Cas9). Commercially available kits can be employed to suppress Helios. A mutation can be made one or more of the protein domains.

Suppression of Helios can increase the number of Th1 polarized T cells in the leukocytes. Suppression of Helios can promote differentiation of T cells to Th1. Suppression of Helios can decrease IL-10, IL-4, or IL-13 expression in the leukocytes. Suppression of Helios can increase IFN-γ expression in the leukocytes. Suppression of Helios can increase IL-12 expression in the leukocytes. Suppression of Helios can decrease the number of Treg polarized T cells in the leukocytes.

Suppression of Helios can increase the population of Th1 cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where Helios is not suppressed. Suppression of Helios can decrease the population of Treg by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where Helios is not suppressed.

Suppression of Helios can increase the expression of one or more related Th1 cell related markers. Suppression of Helios can increase the expression of one or more Th1 cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where Helios is not suppressed. The one or more Th1 related markers can include CCR1, CD4, CD26, CD94, CD1 19, CD183, CD195, CD212, GM-CSF, Granzyme B, IFN-α, IFN-γ, IL-2, IL-12, IL-15, IL-18R, IL-23, IL-27, IL-27R, Lymphotoxin, perforin, t-bet, Tim-3, TNF-α, TRANCE, sCD40L, or any combination thereof. In particular, the one or more Th1 related markers can include IFN-γ, IL-2, IL-12 or any combination thereof. For example, suppression of Helios can increase expression of IFN-γ. For example, suppression of Helios can increase IL-2. For example, suppression of Helios can increase expression of IL-12.

Suppression of Helios can decrease the expression of one or more related Treg cell related markers. Suppression of Helios can decrease the expression of one or more Treg cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where Helios is not suppressed. The one or more Treg related markers can include, TGFβ or IL-10 or any combination thereof. For example, suppression of Helios can decrease TGFβ expression. For example, suppression of Helios can decrease IL-10 expression.

Suppression of Helios can increase the ratio of Th1 T cells to Treg T cells by about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

Suppression of Helios can decrease the ratio of Treg T cells to Th1 T cells by about 1 fold, about 2 fold, about 3 fold, about 4 fold, about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

Suppression of Helios can attenuate exhaustion of CD8+ T cells. Suppression of Helios can decrease CD8+ T cell exhaustion by about 5%, about 10%, about 15%, about 20%, about 25%, about 30%, about 35%, about 40%, about 45%, about 50%, about 60%, about 65%, about 70%, about 75%, about 80%, about 85%, about 90%, about 95% or greater relative to activity without suppression of Helios.

Suppression of Helios can increase the population of CD8+ T cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where Helios is not suppressed.

The activity of Helios can be suppressed (i.e., inhibited) by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater relative to basal activity.

ix. Blimp1

Blimp1 can be suppressed (i.e., inhibited) with a pharmacological agent or by genetic modification. The pharmacological agent could be an inhibitor of Blimp1. The Blimp1 inhibitor can be a small molecule, a small interfering RNA (siRNA), or short hairpin RNA (shRNA). The Blimp1 inhibitor can have a half-maximal inhibitory concentration of less than about 1000 nM, about 900 nM, about 800 mM, about 700 nM, about 600 nM, about 500 nM, about 400 nM, about 300 nM, about 200 nM, about 100 nM or less.

Blimp1 can be suppressed by knocking out the PRDM1 gene from the genome. Techniques for knocking out genes are known by those skilled in the art. Gene knock-out methods in the art, include but are not limited to, gene silencing, conditional knockout, homologous recombination, gene editing, and knockout by mutation. Gene silencing can be achieved using, for example, RNA interference, siRNA or shRNA. Conditional knockout methods can be used to inactivate the PRDM1 gene. A loss of function mutation can help to suppress gene function by creating a mutation in the PRDM1 gene. Gene editing techniques that can be employed to suppress Blimp1 include, but are not limited to, zinc-finger nucleases (ZFN), transcription activator-like effector nucleases (TALEN), meganucleases, and CRISPR-based systems (e.g., CRISPR-Cas9). Commercially available kits can be employed to suppress Blimp1. A mutation can be made one or more of the protein domains.

Suppression of Blimp1 can increase the number of Th1 polarized T cells in the leukocytes. Suppression of Blimp1 can promote differentiation of T cells to Th1. Suppression of Blimp1 can decrease IL-10, IL-4, or IL-13 expression in the leukocytes. Suppression of Blimp1 can increase IFN-γ expression in the leukocytes. Suppression of Blimp1 can increase IL-12 expression in the leukocytes. Suppression of Blimp1 can decrease the number of Treg polarized T cells in the leukocytes.

Suppression of Blimp1 can increase the population of Th1 cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where Blimp1 is not suppressed. Suppression of Blimp1 can decrease the population of Treg by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where Blimp1 is not suppressed.

Suppression of Blimp1 can increase the expression of one or more related Th1 cell related markers. Suppression of Blimp1 can increase the expression of one or more Th1 cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where Blimp1 is not suppressed. The one or more Th1 related markers can include CCR1, CD4, CD26, CD94, CD1 19, CD183, CD195, CD212, GM-CSF, Granzyme B, IFN-α, IFN-γ, IL-2, IL-12, IL-15, IL-18R, IL-23, IL-27, IL-27R, Lymphotoxin, perforin, t-bet, Tim-3, TNF-α, TRANCE, sCD40L, or any combination thereof. In particular, the one or more Th1 related markers can include IFN-γ, IL-2, IL-12 or any combination thereof. For example, suppression of Blimp1 can increase expression of IFN-γ. For example, suppression of Blimp1 can increase IL-2. For example, suppression of Blimp1 can increase expression of IL-12.

Suppression of Blimp1 can decrease the expression of one or more related Treg cell related markers. Suppression of Blimp1 can decrease the expression of one or more Treg cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where Blimp1 is not suppressed. The one or more Treg related markers can include, TGFβ or IL-10 or any combination thereof. For example, suppression of Blimp1 can decrease TGFβ expression. For example, suppression of Blimp1 can decrease IL-10 expression.

Suppression of Blimp1 can increase the ratio of Th1 T cells to Treg T cells by about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

Suppression of Blimp1 can decrease the ratio of Treg T cells to Th1 T cells by about 1 fold, about 2 fold, about 3 fold, about 4 fold, about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

Suppression of Blimp1 can attenuate exhaustion of CD8+ T cells. Suppression of Blimp1 can decrease CD8+ T cell exhaustion by about 5%, about 10%, about 15%, about 20%, about 25%, about 30%, about 35%, about 40%, about 45%, about 50%, about 60%, about 65%, about 70%, about 75%, about 80%, about 85%, about 90%, about 95% or greater relative to activity without suppression of Blimp1.

Suppression of Blimp1 can increase the population of CD8+ T cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where Blimp is not suppressed.

The activity of Blimp1 can be suppressed (i.e., inhibited) by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater relative to basal activity.

x. TOX

TOX (thymocyte selection-associated HMG BOX) can be suppressed (i.e., inhibited) with a pharmacological agent or by genetic modification. The pharmacological agent could be an inhibitor of TOX. The inhibitor can be a small molecule, a small interfering RNA (siRNA), or short hairpin RNA (shRNA). The TOX inhibitor can have a half-maximal inhibitory concentration of less than about 1000 nM, about 900 nM, about 800 mM, about 700 nM, about 600 nM, about 500 nM, about 400 nM, about 300 nM, about 200 nM, about 100 nM or less.

TOX can be suppressed by knocking out the TOX gene from the genome. Techniques for knocking out genes are known by those skilled in the art. Gene knock-out methods in the art, include but are not limited to, gene silencing, conditional knockout, homologous recombination, gene editing, and knockout by mutation. Gene silencing can be achieved using, for example, RNA interference, siRNA or shRNA. Conditional knockout methods can be used to inactivate the TOX gene. A loss of function mutation can help to suppress gene function by creating a mutation in the TOX gene. Gene editing techniques that can be employed to suppress TOX can include, but are not limited to, zinc-finger nucleases (ZFN), transcription activator-like effector nucleases (TALEN), meganucleases, and CRISPR-based systems (e.g., CRISPR-Cas9). Commercially available kits can be employed to suppress TOX. A mutation can be made one or more of the protein domains.

Suppression of TOX can attenuate exhaustion of CD8+ T cells. Suppression of TOX can decrease CD8+ T cell exhaustion by about 5%, about 10%, about 15%, about 20%, about 25%, about 30%, about 35%, about 40%, about 45%, about 50%, about 60%, about 65%, about 70%, about 75%, about 80%, about 85%, about 90%, about 95% or greater relative to activity without suppression of TOX.

Suppression of TOX can increase the population of CD8+ T cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where TOX is not suppressed.

xi. IL-10

IL-10 can be suppressed (i.e., inhibited) with a pharmacological agent or by genetic modification. The pharmacological agent could be an inhibitor of IL-10. The inhibitor can be a small molecule, a small interfering RNA (siRNA), or short hairpin RNA (shRNA). The IL-10 inhibitor can have a half-maximal inhibitory concentration of less than about 1000 nM, about 900 nM, about 800 mM, about 700 nM, about 600 nM, about 500 nM, about 400 nM, about 300 nM, about 200 nM, about 100 nM or less.

IL-10 can be suppressed by knocking out the IL-10 gene from the genome. Techniques for knocking out genes are known by those skilled in the art. Gene knock-out methods in the art, include but are not limited to, gene silencing, conditional knockout, homologous recombination, gene editing, and knockout by mutation. Gene silencing can be achieved using, for example, RNA interference, siRNA or shRNA. Conditional knockout methods can be used to inactivate the IL-10 gene. A loss of function mutation can help to suppress gene function by creating a mutation in the IL-10 gene. Gene editing techniques that can be employed to suppress IL-10 can include, but are not limited to, zinc-finger nucleases (ZFN), transcription activator-like effector nucleases (TALEN), meganucleases, and CRISPR-based systems (e.g., CRISPR-Cas9). Commercially available kits can be employed to suppress IL-10. A mutation can be made one or more of the protein domains.

Suppression of IL-10 can decrease the number of Th2 polarized T cells in the leukocytes. Suppression of IL-10 can increase the number of Th1 polarized T cells in the leukocytes. Suppression of IL-10 can promote differentiation of T cells to Th1. Suppression of IL-10 can decrease IL-10, IL-4, or IL-13 expression in the leukocytes. Suppression of IL-10 can increase IFN-γ expression in the leukocytes. Suppression of IL-10 can increase IL-12 expression in the leukocytes.

Suppression of IL-10 can increase the population of Th1 cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where IL-10 is not suppressed. Suppression of IL-10 can decrease the population of Th2 by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where IL-10 is not suppressed.

Suppression of IL-10 can increase the expression of one or more related Th1 cell related markers. Suppression of IL-10 can increase the expression of one or more Th1 cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where IL-10 is not suppressed. The one or more Th1 related markers can include CCR1, CD4, CD26, CD94, CD1 19, CD183, CD195, CD212, GM-CSF, Granzyme B, IFN-α, IFN-γ, IL-2, IL-12, IL-15, IL-18R, IL-23, IL-27, IL-27R, Lymphotoxin, perforin, t-bet, Tim-3, TNF-α, TRANCE, sCD40L, or any combination thereof. In particular, the one or more Th1 related markers can include IFN-γ, IL-2, IL-12 or any combination thereof. For example, suppression of IL-10 can increase expression of IFN-γ. For example, suppression of IL-10 can increase IL-2. For example, suppression of IL-10 can increase expression of IL-12.

Suppression of IL-10 can decrease the expression of one or more related Th2 cell related markers. Suppression of IL-10 can decrease the expression of one or more Th2 cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where IL-10 is not suppressed. The one or more Th2 related markers can include CCR3, CCR4, CCR7, CCR8, CD4, CD30, CD81, CD184, CD278, c-maf, CRTH2, Gata-3, GM-CSF, IFN yR, IgD, IL-1R, IL-4, IL-5, IL-6, IL-9, IL-10, IL-13, IL-15, ST2L/T1, Tim-1, or any combination thereof. In particular, the one or more Th2 related markers can include IL-4, IL-6, IL-10, IL-13, IL-15 or any combination thereof. For example, suppression of IL-10 can decease IL-4 expression. For example, suppression of IL-10 can decrease IL-5 expression. For example, suppression of IL-10 can decrease IL-6 expression. For example, suppression of IL-10 can decrease IL-10 expression. For example, suppression of IL-10 can decrease IL-13 expression. For example, suppression of IL-10 can decrease IL-15 expression.

Suppression of IL-10 can increase the ratio of Th1 T cells to Th2 T cells by about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

Suppression of IL-10 can decrease the ratio of Th2 T cells to Th1 T cells by about 1 fold, about 2 fold, about 3 fold, about 4 fold, about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

The activity of IL-10 can be suppressed (i.e., inhibited) by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater relative to basal activity.

Suppression of IL-10 can encourage inflammation and production of pro-inflammatory cytokines in other T cells. Suppression of IL-10 can increase expression of IL-1, IL-12, IL-18, TNF-alpha, IFN-gamma, or GM-CSF.

Suppression of IL-10 can increase the expression of IL-1 by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where IL-10 is not suppressed.

Suppression of IL-10 can increase the expression of IL-12 by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where IL-10 is not suppressed.

Suppression of IL-10 can increase the expression of IL-18 by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where IL-10 is not suppressed.

Suppression of IL-10 can increase the expression of TFN-alpha by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where IL-10 is not suppressed.

Suppression of IL-10 can increase the expression of I by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where IL-10 is not suppressed.

Suppression of IL-10 can increase the expression of IL-18 by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where IL-10 is not suppressed.

Suppression of IL-10 can increase the expression of GM-CFS by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where IL-10 is not suppressed.

xii. SOCS1

SOCS1 can be suppressed (i.e., inhibited) with a pharmacological agent or by genetic modification. The pharmacological agent could be an inhibitor of SOCS1. The SOCS1 inhibitor can be a small molecule, a small interfering RNA (siRNA), or short hairpin RNA (shRNA). The SOCS1 inhibitor can have a half-maximal inhibitory concentration of less than about 1000 nM, about 900 nM, about 800 mM, about 700 nM, about 600 nM, about 500 nM, about 400 nM, about 300 nM, about 200 nM, about 100 nM or less.

SOCS1 can be suppressed by knocking out the SOCS1 gene from the genome. Techniques for knocking out genes are known by those skilled in the art. Gene knock-out methods in the art include, but are not limited to, gene silencing, conditional knockout, homologous recombination, gene editing, and knockout by mutation. Gene silencing can be achieved using, for example, RNA interference, siRNA or shRNA. Conditional knockout methods can be used to inactivate the SOCS1 gene. A loss of function mutation can help to suppress gene function by creating a mutation in the SOCS1 gene. Gene editing techniques that can be employed to suppress SOCS1 include, but are not limited to, zinc-finger nucleases (ZFN), transcription activator-like effector nucleases (TALEN), meganucleases, and CRISPR-based systems (e.g., CRISPR-Cas9). Commercially available kits can be employed to suppress SOCS1. A mutation can be made one or more of the protein domains.

Suppression of SOCS1 can increase the number of Th1 polarized T cells in the leukocytes. Suppression of SOCS1 can promote differentiation of T cells to Th1. Suppression of SOCS1 can decrease IL-10, IL-4, or IL-13 expression in the leukocytes. Suppression of SOCS1 can increase IFN-γ expression in the leukocytes. Suppression of SOCS1 can increase IL-12 expression in the leukocytes. Suppression of SOCS1 can decrease the number of Treg polarized T cells in the leukocytes.

Suppression of SOCS1 can increase the population of Th1 cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where SOCS1 is not suppressed. Suppression of SOCS1 can decrease the population of Treg by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where SOCS1 is not suppressed.

Suppression of SOCS1 can increase the expression of one or more related Th1 cell related markers. Suppression of SOCS1 can increase the expression of one or more Th1 cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where SOCS1 is not suppressed. The one or more Th1 related markers can include CCR1, CD4, CD26, CD94, CD1 19, CD183, CD195, CD212, GM-CSF, Granzyme B, IFN-α, IFN-γ, IL-2, IL-12, IL-15, IL-18R, IL-23, IL-27, IL-27R, Lymphotoxin, perforin, t-bet, Tim-3, TNF-α, TRANCE, sCD40L, or any combination thereof. In particular, the one or more Th1 related markers can include IFN-γ, IL-2, IL-12 or any combination thereof. For example, suppression of SOCS1 can increase expression of IFN-γ. For example, suppression of SOCS1 can increase IL-2. For example, suppression of SOCS1 can increase expression of IL-12.

Suppression of SOCS1 can decrease the expression of one or more related Treg cell related markers. Suppression of SOCS1 can decrease the expression of one or more Treg cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where SOCS1 is not suppressed. The one or more Treg related markers can include, TGFβ or IL-10 or any combination thereof. For example, suppression of SOCS1 can decrease TGFβ expression. For example, suppression of SOCS1 can decrease IL-10 expression.

Suppression of SOCS1 can increase the ratio of Th1 T cells to Treg T cells by about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

Suppression of SOCS1 can decrease the ratio of Treg T cells to Th1 T cells by about 1 fold, about 2 fold, about 3 fold, about 4 fold, about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

Suppression of SOCS1 can attenuate exhaustion of CD8+ T cells. Suppression of SOCS1 can decrease CD8+ T cell exhaustion by about 5%, about 10%, about 15%, about 20%, about 25%, about 30%, about 35%, about 40%, about 45%, about 50%, about 60%, about 65%, about 70%, about 75%, about 80%, about 85%, about 90%, about 95% or greater relative to activity without suppression of SOCS1.

Suppression of SOCS1 can increase the population of CD8+ T cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where SOCS1 is not suppressed.

The activity of SOCS1 can be suppressed (i.e., inhibited) by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater relative to basal activity.

xiii. PD-1

PD-1 can be suppressed (i.e., inhibited) with a pharmacological agent or by genetic modification. The pharmacological agent could be an inhibitor of PD-1. The PD-1 inhibitor can be a small molecule, a small interfering RNA (siRNA), or short hairpin RNA (shRNA). The PD-1 inhibitor can have a half-maximal inhibitory concentration of less than about 1000 nM, about 900 nM, about 800 mM, about 700 nM, about 600 nM, about 500 nM, about 400 nM, about 300 nM, about 200 nM, about 100 nM or less.

PD-1 can be suppressed by knocking out the PD-1 gene from the genome. Techniques for knocking out genes are known by those skilled in the art. Gene knock-out methods in the art include, but are not limited to, gene silencing, conditional knockout, homologous recombination, gene editing, and knockout by mutation. Gene silencing can be achieved using, for example, RNA interference, siRNA or shRNA. Conditional knockout methods can be used to inactivate the PD-1 gene. A loss of function mutation can help to suppress gene function by creating a mutation in the PD-1 gene. Gene editing techniques that can be employed to suppress PD-1 include, but are not limited to, zinc-finger nucleases (ZFN), transcription activator-like effector nucleases (TALEN), meganucleases, and CRISPR-based systems (e.g., CRISPR-Cas9). Commercially available kits can be employed to suppress PD-1. A mutation can be made one or more of the protein domains.

Suppression of PD-1 can increase the number of Th1 polarized T cells in the leukocytes. Suppression of PD-1 can promote differentiation of T cells to Th1. Suppression of PD-1 can decrease IL-10, IL-4, or IL-13 expression in the leukocytes. Suppression of PD-1 can increase IFN-γ expression in the leukocytes. Suppression of PD-1 can increase IL-12 expression in the leukocytes. Suppression of PD-1 can decrease the number of Treg polarized T cells in the leukocytes.

Suppression of PD-1 can increase the population of Th1 cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where PD-1 is not suppressed. Suppression of PD-1 can decrease the population of Treg by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where PD-1 is not suppressed.

Suppression of PD-1 can increase the expression of one or more related Th1 cell related markers. Suppression of PD-1 can increase the expression of one or more Th1 cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where PD-1 is not suppressed. The one or more Th1 related markers can include CCR1, CD4, CD26, CD94, CD1 19, CD183, CD195, CD212, GM-CSF, Granzyme B, IFN-α, IFN-γ, IL-2, IL-12, IL-15, IL-18R, IL-23, IL-27, IL-27R, Lymphotoxin, perforin, t-bet, Tim-3, TNF-α, TRANCE, sCD40L, or any combination thereof. In particular, the one or more Th1 related markers can include IFN-γ, IL-2, IL-12 or any combination thereof. For example, suppression of PD-1 can increase expression of IFN-γ. For example, suppression of PD-1 can increase IL-2. For example, suppression of PD-1 can increase expression of IL-12.

Suppression of PD-1 can decrease the expression of one or more related Treg cell related markers. Suppression of PD-1 can decrease the expression of one or more Treg cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where PD-1 is not suppressed. The one or more Treg related markers can include, TGFβ or IL-10 or any combination thereof. For example, suppression of PD-1 can decrease TGFβ expression. For example, suppression of PD-1 can decrease IL-10 expression.

Suppression of PD-1 can increase the ratio of Th1 T cells to Treg T cells by about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

Suppression of PD-1 can decrease the ratio of Treg T cells to Th1 T cells by about 1 fold, about 2 fold, about 3 fold, about 4 fold, about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

Suppression of PD-1 can attenuate exhaustion of CD8+ T cells. Suppression of PD-1 can decrease CD8+ T cell exhaustion by about 5%, about 10%, about 15%, about 20%, about 25%, about 30%, about 35%, about 40%, about 45%, about 50%, about 60%, about 65%, about 70%, about 75%, about 80%, about 85%, about 90%, about 95% or greater relative to activity without suppression of PD-1.

Suppression of PD-1 can increase the population of CD8+ T cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where PD-1 is not suppressed.

The activity of PD-1 can be suppressed (i.e., inhibited) by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater relative to basal activity.

xiv. LAG-3

LAG-3 can be suppressed (i.e., inhibited) with a pharmacological agent or by genetic modification. The pharmacological agent could be an inhibitor of LAG-3. The inhibitor can be a small molecule, a small interfering RNA (siRNA), or short hairpin RNA (shRNA). The LAG-3 inhibitor can have a half-maximal inhibitory concentration of less than about 1000 nM, about 900 nM, about 800 mM, about 700 nM, about 600 nM, about 500 nM, about 400 nM, about 300 nM, about 200 nM, about 100 nM or less.

LAG-3 can be suppressed by knocking out the LAG-3 gene from the genome. Techniques for knocking out genes are known by those skilled in the art. Gene knock-out methods in the art, include but are not limited to, gene silencing, conditional knockout, homologous recombination, gene editing, and knockout by mutation. Gene silencing can be achieved using, for example, RNA interference, siRNA or shRNA. Conditional knockout methods can be used to inactivate the LAG-3 gene. A loss of function mutation can help to suppress gene function by creating a mutation in the LAG-3 gene. Gene editing techniques that can be employed to suppress LAG-3 can include, but are not limited to, zinc-finger nucleases (ZFN), transcription activator-like effector nucleases (TALEN), meganucleases, and CRISPR-based systems (e.g., CRISPR-Cas9). Commercially available kits can be employed to suppress LAG-3. A mutation can be made one or more of the protein domains.

Suppression of LAG-3 can increase the number of Th1 polarized T cells in the leukocytes. Suppression of LAG-3 can promote differentiation of T cells to Th1. Suppression of LAG-3 can decrease IL-10, IL-4, or IL-13 expression in the leukocytes. Suppression of LAG-3 can increase IFN-γ expression in the leukocytes. Suppression of LAG-3 can increase IL-12 expression in the leukocytes. Suppression of LAG-3 can decrease the number of Treg polarized T cells in the leukocytes.

Suppression of LAG-3 can increase the population of Th1 cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where LAG-3 is not suppressed. Suppression of LAG-3 can decrease the population of Treg cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where LAG-3 is not suppressed.

Suppression of LAG-3 can increase the expression of one or more related Th1 cell related markers. Suppression of LAG-3 can increase the expression of one or more Th1 cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where LAG-3 is not suppressed. The one or more Th1 related markers can include CCR1, CD4, CD26, CD94, CD1 19, CD183, CD195, CD212, GM-CSF, Granzyme B, IFN-α, IFN-γ, IL-2, IL-12, IL-15, IL-18R, IL-23, IL-27, IL-27R, Lymphotoxin, perforin, t-bet, Tim-3, TNF-α, TRANCE, sCD40L, or any combination thereof. In particular, the one or more Th1 related markers can include IFN-γ, IL-2, IL-12 or any combination thereof. For example, suppression of LAG-3 can increase expression of IFN-γ. For example, suppression of LAG-3 can increase IL-2. For example, suppression of LAG-3 can increase expression of IL-12.

Suppression of LAG-3 can decrease the expression of one or more related Treg cell related markers. Suppression of LAG-3 can decrease the expression of one or more Treg cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where LAG-3 is not suppressed. The one or more Treg related markers can include, TGFβ or IL-10 or any combination thereof. For example, suppression of LAG-3 can decrease TGFβ expression. For example, suppression of LAG-3 can decrease IL-10 expression.

Suppression of LAG-3 can increase the ratio of Th1 T cells to Treg T cells by about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

Suppression of LAG-3 can decrease the ratio of Treg T cells to Th1 T cells by about 1 fold, about 2 fold, about 3 fold, about 4 fold, about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

The activity of LAG-3 can be suppressed (i.e., inhibited) by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater relative to basal activity.

xv. TNF-Alpha

TNF-alpha can be suppressed (i.e., inhibited) with a pharmacological agent or by genetic modification. The pharmacological agent could be an inhibitor of TNF-alpha. The TNF-alpha inhibitor can be a small molecule, a small interfering RNA (siRNA), or short hairpin RNA (shRNA). The TNF-alpha inhibitor can have a half-maximal inhibitory concentration of less than about 1000 nM, about 900 nM, about 800 mM, about 700 nM, about 600 nM, about 500 nM, about 400 nM, about 300 nM, about 200 nM, about 100 nM or less.

TNF-alpha can be suppressed by knocking out the TNF-alpha gene from the genome. Techniques for knocking out genes are known by those skilled in the art. Gene knock-out methods in the art include, but are not limited to, gene silencing, conditional knockout, homologous recombination, gene editing, and knockout by mutation. Gene silencing can be achieved using, for example, RNA interference, siRNA or shRNA. Conditional knockout methods can be used to inactivate the TNF-alpha gene. A loss of function mutation can help to suppress gene function by creating a mutation in the TNF-alpha gene. Gene editing techniques that can be employed to suppress TNF-alpha include, but are not limited to, zinc-finger nucleases (ZFN), transcription activator-like effector nucleases (TALEN), meganucleases, and CRISPR-based systems (e.g., CRISPR-Cas9). Commercially available kits can be employed to suppress TNF-alpha. A mutation can be made one or more of the protein domains.

Suppression of TNF-alpha can increase the number of Th1 polarized T cells in the leukocytes. Suppression of TNF-alpha can promote differentiation of T cells to Th1. Suppression of TNF-alpha can decrease IL-10, IL-4, or IL-13 expression in the leukocytes. Suppression of TNF-alpha can increase IFN-γ expression in the leukocytes. Suppression of TNF-alpha can increase IL-12 expression in the leukocytes. Suppression of TNF-alpha can decrease the number of Treg polarized T cells in the leukocytes.

Suppression of TNF-alpha can increase the population of Th1 cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where TNF-alpha is not suppressed. Suppression of TNF-alpha can decrease the population of Treg by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where TNF-alpha is not suppressed.

Suppression of TNF-alpha can increase the expression of one or more related Th1 cell related markers. Suppression of TNF-alpha can increase the expression of one or more Th1 cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where TNF-alpha is not suppressed. The one or more Th1 related markers can include CCR1, CD4, CD26, CD94, CD1 19, CD183, CD195, CD212, GM-CSF, Granzyme B, IFN-α, IFN-γ, IL-2, IL-12, IL-15, IL-18R, IL-23, IL-27, IL-27R, Lymphotoxin, perforin, t-bet, Tim-3, TNF-α, TRANCE, sCD40L, or any combination thereof. In particular, the one or more Th1 related markers can include IFN-γ, IL-2, IL-12 or any combination thereof. For example, suppression of TNF-alpha can increase expression of IFN-γ. For example, suppression of TNF-alpha can increase IL-2. For example, suppression of TNF-alpha can increase expression of IL-12.

Suppression of TNF-alpha can decrease the expression of one or more related Treg cell related markers. Suppression of TNF-alpha can decrease the expression of one or more Treg cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where TNF-alpha is not suppressed. The one or more Treg related markers can include, TGFβ or IL-10 or any combination thereof. For example, suppression of TNF-alpha can decrease TGFβ expression. For example, suppression of TNF-alpha can decrease IL-10 expression.

Suppression of TNF-alpha can increase the ratio of Th1 T cells to Treg T cells by about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

Suppression of TNF-alpha can decrease the ratio of Treg T cells to Th1 T cells by about 1 fold, about 2 fold, about 3 fold, about 4 fold, about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

Suppression of TNF-alpha can attenuate exhaustion of CD8+ T cells. Suppression of TNF-alpha can decrease CD8+ T cell exhaustion by about 5%, about 10%, about 15%, about 20%, about 25%, about 30%, about 35%, about 40%, about 45%, about 50%, about 60%, about 65%, about 70%, about 75%, about 80%, about 85%, about 90%, about 95% or greater relative to activity without suppression of TNF-alpha.

Suppression of TNF-alpha can increase the population of CD8+ T cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where TNF-alpha is not suppressed.

The activity of TNF-alpha can be suppressed (i.e., inhibited) by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater relative to basal activity.

Cytokine release syndrome is a known complication of the treatment of hematologic malignancies with chimeric antigen receptor-modified (CAR) T cells or with T cell replete, HLA-haploidentical blood or marrow transplantation. In embodiments, inhibition of TNF-alpha can attenuate cytokine release syndrome after non-engrafting, CD8-depleted donor lymphocyte infusion.

Cytokine release syndrome is graded on a scale from 0 to 5. Suppression of TNF-alpha can decrease the cytokine release syndrome score to 0, 1, 2, 3, or 4.

Suppression of TNF-alpha can decrease the expression of IL-10 by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where TNF-alpha is not suppressed.

Suppression of TNF-alpha can decrease the percentage of pyroptotic leukocytes among total leukocytes by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where TNF-alpha is not suppressed.

The activity of TNF-alpha, as measured for example by phosphorylation of one of its substrates (such as 1-Phosphatidylinositol-4,5-bisphosphate phosphodiesterase gamma-2; PLC-γ2) can be suppressed (i.e., inhibited) by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater relative to basal activity.

xvi. TGF-Beta Receptor II

TGF-beta Receptor II can be suppressed (i.e., inhibited) with a pharmacological agent or by genetic modification. The pharmacological agent could be an inhibitor of TGF-beta Receptor II. The TGF-beta Receptor II inhibitor can be a small molecule, a small interfering RNA (siRNA), or short hairpin RNA (shRNA). The TGF-beta Receptor II inhibitor can have a half-maximal inhibitory concentration of less than about 1000 nM, about 900 nM, about 800 mM, about 700 nM, about 600 nM, about 500 nM, about 400 nM, about 300 nM, about 200 nM, about 100 nM or less.

TGF-beta Receptor II can be suppressed by knocking out the TGF-beta Receptor II gene from the genome. Techniques for knocking out genes are known by those skilled in the art. Gene knock-out methods in the art include, but are not limited to, gene silencing, conditional knockout, homologous recombination, gene editing, and knockout by mutation. Gene silencing can be achieved using, for example, RNA interference, siRNA or shRNA. Conditional knockout methods can be used to inactivate the TGF-beta Receptor II gene. A loss of function mutation can help to suppress gene function by creating a mutation in the TGF-beta Receptor II gene. Gene editing techniques that can be employed to suppress TGF-beta Receptor II include, but are not limited to, zinc-finger nucleases (ZFN), transcription activator-like effector nucleases (TALEN), meganucleases, and CRISPR-based systems (e.g., CRISPR-Cas9). Commercially available kits can be employed to suppress TGF-beta Receptor II. A mutation can be made one or more of the protein domains.

Suppression of TGF-beta Receptor II can increase the number of Th1 polarized T cells in the leukocytes. Suppression of TGF-beta Receptor II can promote differentiation of T cells to Th1. Suppression of TGF-beta Receptor II can decrease IL-10, IL-4, or IL-13 expression in the leukocytes. Suppression of TGF-beta Receptor II can increase IFN-γ expression in the leukocytes. Suppression of TGF-beta Receptor II can increase IL-12 expression in the leukocytes. Suppression of TGF-beta Receptor II can decrease the number of Treg polarized T cells in the leukocytes.

Suppression of TGF-beta Receptor II can increase the population of Th1 cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where TGF-beta Receptor II is not suppressed. Suppression of TGF-beta Receptor II can decrease the population of Treg by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where TGF-beta Receptor II is not suppressed.

Suppression of TGF-beta Receptor II can increase the expression of one or more related Th1 cell related markers. Suppression of TGF-beta Receptor II can increase the expression of one or more Th1 cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where TGF-beta Receptor II is not suppressed. The one or more Th1 related markers can include CCR1, CD4, CD26, CD94, CD1 19, CD183, CD195, CD212, GM-CSF, Granzyme B, IFN-α, IFN-γ, IL-2, IL-12, IL-15, IL-18R, IL-23, IL-27, IL-27R, Lymphotoxin, perforin, t-bet, Tim-3, TNF-α, TRANCE, sCD40L, or any combination thereof. In particular, the one or more Th1 related markers can include IFN-7, IL-2, IL-12 or any combination thereof. For example, suppression of TGF-beta Receptor II can increase expression of IFN-γ. For example, suppression of TGF-beta Receptor II can increase IL-2. For example, suppression of TGF-beta Receptor II can increase expression of IL-12.

Suppression of TGF-beta Receptor II can decrease the expression of one or more related Treg cell related markers. Suppression of TGF-beta Receptor II can decrease the expression of one or more Treg cell related markers by about 1%, about 2%, about 3%, about 4%, about 5%, about 6%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where TGF-beta Receptor II is not suppressed. The one or more Treg related markers can include, TGFβ or IL-10 or any combination thereof. For example, suppression of TGF-beta Receptor II can decrease TGFβ expression. For example, suppression of TGF-beta Receptor II can decrease IL-10 expression.

Suppression of TGF-beta Receptor II can increase the ratio of Th1 T cells to Treg T cells by about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

Suppression of TGF-beta Receptor II can decrease the ratio of Treg T cells to Th1 T cells by about 1 fold, about 2 fold, about 3 fold, about 4 fold, about 5 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 150 fold, about 200 fold, about 250 fold, about 300 fold, about 350 fold, about 400 fold, about 450 fold, about 500 fold, about 550 fold, about 600 fold, about 650 fold, about 700 fold, about 750 fold, about 800 fold, about 850 fold, about 900 fold, about 950 fold, about 1000 fold or greater.

Suppression of TGF-beta Receptor II can attenuate exhaustion of CD8+ T cells. Suppression of TGF-beta Receptor II can decrease CD8+ T cell exhaustion by about 5%, about 10%, about 15%, about 20%, about 25%, about 30%, about 35%, about 40%, about 45%, about 50%, about 60%, about 65%, about 70%, about 75%, about 80%, about 85%, about 90%, about 95% or greater relative to activity without suppression of TGF-beta Receptor II.

Suppression of TGF-beta Receptor II can increase the population of CD8+ T cells by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater compared to leukocytes where TGF-beta Receptor II is not suppressed.

The activity of TGF-beta Receptor II can be suppressed (i.e., inhibited) by about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater relative to basal activity.

### C. Therapeutic Applications

The disclosure also relates to methods for treating a disease or condition, such as cancer. The method comprises administering to a subject in need thereof a lymphodepleting agent and/or an immune-stimulating agent and administering to the subject an allogenic lymphocyte composition as described herein.

The lymphodepleting agent can be a cytoreductive agent. Exemplary cytoreductive agents include, but are not limited to, an alkylating agent, alkyl sulphonates, nitrosoureas, triazene, antimetabolites, pyrimidine analog, purine analog, vinca alkaloids, epiodophyllotoxins, antibiotics, dirbromannitol, deoxyspergualine, dimethyl myleran and tiotepa.

The lymphodepleting agent can be a chemotherapeutic agent or a biologic agent. Exemplary chemotherapeutic agents and/or biologic agents include, but are not limited to, an antibody, a B cell receptor pathway inhibitor, a T cell receptor inhibitor, a PI3K inhibitor, an IAP inhibitor, an mTOR inhibitor, a radioimmunotherapeutic, a DNA damaging agent, a histone deacetylase inhibitor, a protein kinase inhibitor, a hedgehog inhibitor, an Hsp90 inhibitor, a telomerase inhibitor, a Jak1/2 inhibitor, a protease inhibitor, an IRAK inhibitor, a PKC inhibitor, a PARP inhibitor, a CYP3 A4 inhibitor, an AKT inhibitor, an Erk inhibitor, a proteosome inhibitor, an alkylating agent, an anti-metabolite, a plant alkaloid, a terpenoid, a cytotoxin, a topoisomerase inhibitor, a CD79A inhibitor, a CD79B inhibitor, a CD 19 inhibitor, a Lyn inhibitor, a Syk inhibitor, a PI3K inhibitor, a Blnk inhibitor, a PLCy inhibitor, a PKCP inhibitor, a CD22 inhibitor, a Bcl-2 inhibitor, an IRAK 1/4 inhibitor, a JAK inhibitor (e.g., ruxolitinib, baricitinib, CYT387, lestauritinib, pacritinib, TG101348, SAR302503, tofacitinib (Xeljanz), etanercept (Enbrel), GLPG0634, R256), a microtubule inhibitor, a Topo II inhibitor, anti-TWEAK antibody, anti-IL17 bispecific antibody, a CK2 inhibitor, anaplastic lymphoma kinase (ALK) and c-Met inhibitors, demethylase enzyme inhibitors such as demethylase, HDM, LSDI and KDM, fatty acid synthase inhibitors such as spirocyclic piperidine derivatives, glucocorticosteriod receptor agonist, fusion anti-CD 19-cytotoxic agent conjugate, antimetabolite, p70S6K inhibitor, immune modulators, AKT/PKB inhibitor, procaspase-3 activator PAC-1, BRAF inhibitor, lactate dehydrogenase A (LDH-A) inhibitor, CCR2 inhibitor, CXCR4 inhibitor, chemokine receptor antagonists, DNA double stranded break repair inhibitors, NOR202, GA-101, TLR2 inhibitor, Muromonab-CD3, rituximab (rituxan), carfilzomib, fludarabine, cyclophosphamide, vincristine, chlorambucil, ifosphamide, doxorubicin, mesalazine, thalidomide, revlimid, lenalidomide, temsirolimus, everolimus, fostamatinib, paclitaxel, docetaxel, ofatumumab, dexamethasone, bendamustine, CAL-101, ibritumomab, tositumomab, bortezomib, pentostatin, endostatin, ritonavir, ketoconazole, an anti-VEGF antibody, herceptin, cetuximab, cisplatin, carboplatin, docetaxel, erlotinib, etopiside, 5-fluorouracil, gemcitabine, ifosphamide, imatinib mesylate (Gleevec), gefitinib, erlotinib, procarbazine, irinotecan, leucovorin, mechlorethamine, methotrexate, oxaliplatin, paclitaxel, sorafenib, sunitinib, topotecan, vinblastine, GA-1101, dasatinib, Sipuleucel-T, disulfiram, epigallocatechin-3-gallate, salinosporamide A, ONX0912, CEP-18770, MLN9708, R-406, lenalinomide, spirocyclic piperidine derivatives, quinazoline carboxamide azetidine compounds, thiotepa, DWA2114R, NK121, IS 3 295, 254-5, alkyl sulfonates such as busulfan, improsulfan and piposulfan, aziridines such as benzodepa, carboquone, meturedepa and uredepa, ethylenimine, methylmelamines such as altretamine, triethylenemelamine, triethylenephosphoramide, triethylenethiophosphoramide and trimethylmelamine, chlornaphazine, estramustine, ifosfamide, mechlorethamine, oxide hydrochloride, novobiocin, phenesterine, prednimustine, trofosfamide, uracil mustard, nitrosoureas such as carmustine, chlorozotocin, fotemustine, lomustine, nimustine, ranimustine, antibiotics such as aclacinomycins, actinomycin, anthramycin, azaserine, bleomycins, cactinomycin, calicheamicin, carubicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin, antimetabolites such as methotrexate and 5-fluorouracil (5-FU), folic acid analogues such as denopterin, methotrexate, pteropterin, trimetrexate, purine analogs such as fludarabine, 6-mercaptopurine, thiamiprine, thioguanine, pyrimidine analogs such as ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone, anti-adrenals such as aminoglutethimide, mitotane, trilostane, folic acid replenisher such as folinic acid, aceglatone, aldophosphamide glycoside, aminolevulinic acid, amsacrine, bestrabucil, bisantrene, edatrexate, defosfamide, demecolcine, diaziquone, eflornithine, elliptinium acetate, etoglucid, gallium nitrate, hydroxyurea, lentinan, lonidamine, mitoguazone, mitoxantrone, mopidamol, nitracrine, pentostatin, phenamet, pirarubicin, podophyllinic acid, 2-ethylhydrazide, procarbazine, polysaccharide-K, razoxane, sizofiran, spirogermanium, tenuazonic acid, triaziquone, 2, 2′,2″-trichlorotriethylamine, urethan, vindesine, dacarbazine, mannomustine, mitobronitol, mitolactol, pipobroman, gacytosine, cytosine arabinoside, taxoids, e.g., paclitaxel and docetaxel, 6-thioguanine, mercaptopurine, methotrexate, platinum analogs, platinum, etoposide (VP-16), ifosfamide, mitomycin C, mitoxantrone, vincristine, vinorelbine, Navelbine, Novantrone, teniposide, daunomycin, aminopterin, Xeloda, ibandronate, CPT1 1, topoisomerase inhibitor RFS 2000, difluoromethylornithine (DMFO), retinoic acid, esperamycins, capecitabine, and pharmaceutically acceptable salts, acids or derivatives of, anti-hormonal agents such as anti-estrogens including for example tamoxifen, raloxifene, aromatase inhibiting 4(5)-imidazoles, 4-hydroxytamoxifen, trioxifene, keoxifene, LY117018, onapristone and toremifene (Fareston), antiandrogens such as flutamide, nilutamide, bicalutamide, leuprolide and goserelin, ACK inhibitors such as AVL-263 (Avila Therapeutics/Celgene Corporation), AVL-292 (Avila Therapeutics/Celgene Corporation), AVL-291 (Avila Therapeutics/Celgene Corporation), BMS-488516 (Bristol-Myers Squibb), BMS-509744 (Bristol-Myers Squibb), CGI-1746 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) or a combination thereof.

The compositions and methods disclosed herein can used for any suitable cancer, including, but not limited to, bladder cancer, brain cancer, breast cancer, colorectal cancer, cervical cancer, gastrointestinal cancer, genitourinary cancer, head and neck cancer, lung cancer, ovarian cancer, prostate cancer, renal cancer, skin cancer, and testicular cancer, cardiac cancers, including, for example sarcoma, e.g., angiosarcoma, fibrosarcoma, rhabdomyosarcoma, and liposarcoma, myxoma, rhabdomyoma, fibroma, lipoma and teratoma, lung cancers, including, for example, bronchogenic carcinoma, e.g., squamous cell, undifferentiated small cell, undifferentiated large cell, and adenocarcinoma, alveolar and bronchiolar carcinoma, bronchial adenoma, sarcoma, lymphoma, chondromatous hamartoma, and mesothelioma, gastrointestinal cancer, including, for example, cancers of the esophagus, e.g., squamous cell carcinoma, adenocarcinoma, leiomyosarcoma, and lymphoma, cancers of the stomach, e.g., carcinoma, lymphoma, and leiomyosarcoma, cancers of the pancreas, e.g., ductal adenocarcinoma, insulinoma, glucagonoma, gastrinoma, carcinoid tumors, and vipoma, cancers of the small bowel, e.g., adenocarcinoma, lymphoma, carcinoid tumors, Kaposi's sarcoma, leiomyoma, hemangioma, lipoma, neurofibroma, and fibroma, cancers of the large bowel, e.g., adenocarcinoma, tubular adenoma, villous adenoma, hamartoma, and leiomyoma, genitourinary tract cancers, including, for example, cancers of the kidney, e.g., adenocarcinoma, Wilm's tumor (nephroblastoma), lymphoma, and leukemia, cancers of the bladder and urethra, e.g., squamous cell carcinoma, transitional cell carcinoma, and adenocarcinoma, cancers of the prostate, e.g., adenocarcinoma, and sarcoma, cancer of the testis, e.g., seminoma, teratoma, embryonal carcinoma, teratocarcinoma, choriocarcinoma, sarcoma, interstitial cell carcinoma, fibroma, fibroadenoma, adenomatoid tumors, and lymphoma, liver cancers, including, for example, hepatoma, e.g., hepatocellular carcinoma, cholangiocarcinoma, hepatoblastoma, angiosarcoma, hepatocellular adenoma, and hemangioma, bone cancers, including, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochrondroma (osteocartilaginous exostoses), benign chondroma, chondroblastoma, chondromyxofibroma, osteoid osteoma and giant cell tumors, nervous system cancers, including, for example, cancers of the skull, e.g., osteoma, hemangioma, granuloma, xanthoma, and osteitis defoinians, cancers of the meninges, e.g., meningioma, meningiosarcoma, and gliomatosis, cancers of the brain, e.g., astrocytoma, medulloblastoma, glioma, ependymoma, germinoma (pinealoma), glioblastoma multiform, oligodendroglioma, schwannoma, retinoblastoma, and congenital tumors, and cancers of the spinal cord, e.g., neurofibroma, meningioma, glioma, and sarcoma, gynecological cancers, including, for example, cancers of the uterus, e.g., endometrial carcinoma, cancers of the cervix, e.g., cervical carcinoma, and pre tumor cervical dysplasia, cancers of the ovaries, e.g., ovarian carcinoma, including serous cystadenocarcinoma, mucinous cystadenocarcinoma, unclassified carcinoma, granulosa thecal cell tumors, Sertoli Leydig cell tumors, dysgerminoma, and malignant teratoma, cancers of the vulva, e.g., squamous cell carcinoma, intraepithelial carcinoma, adenocarcinoma, fibrosarcoma, and melanoma, cancers of the vagina, e.g., clear cell carcinoma, squamous cell carcinoma, botryoid sarcoma, and embryonal rhabdomyosarcoma, and cancers of the fallopian tubes, e.g., carcinoma, hematologic cancers, including, for example, cancers of the blood, e.g., acute myeloid leukemia, chronic myeloid leukemia, acute lymphoblastic leukemia, chronic lymphocytic leukemia, myeloproliferative diseases, multiple myeloma, and myelodysplasia syndrome, Hodgkin's lymphoma, non-Hodgkin's lymphoma (malignant lymphoma) and Waldenstrom's macro globulinemia, skin cancers, including, for example, malignant melanoma, basal cell carcinoma, squamous cell carcinoma, Kaposi's sarcoma, moles dysplastic nevi, lipoma, angioma, dermatofibroma, keloids, psoriasis, and adrenal gland cancers, including, for example, neuroblastoma. In certain embodiments, when the disease is cancer, it may include a lung cancer tumor, a breast cancer tumor, a prostate cancer tumor, a brain cancer tumor, or a skin cancer tumor for example.

The subject can have a solid tumor. In some embodiments, the subject can have a sarcoma, carcinoma, or a neurofibromatoma. In some embodiments, the subject can have a colon cancer. In some embodiments, the subject can have a lung cancer. In some embodiments, the subject can have an ovarian cancer. In some embodiments, the subject can have a pancreatic cancer. In some embodiments, the subject can have a prostate cancer. In some embodiments, the subject can have a proximal or distal bile duct carcinoma. In some embodiments, the subject can have a breast cancer. In some embodiments, the subject can have a HER2-positive breast cancer. In some embodiments, the subject can have a HER2-negative breast cancer. In some embodiments, the subject has been treated for a solid tumor, and the method is applied to treat a subject as adjuvant therapy, that is the method is applied to the subject when the cancer is in a complete remission so as to prevent relapse of the cancer.

The subject can have a hematologic cancer. In some embodiments, the cancer is a leukemia, a lymphoma, a myeloma, a myelodysplastic syndrome, or a myeloproliferative neoplasm. In some embodiments, the cancer is a non-Hodgkin lymphoma. In some embodiments, the cancer is a Hodgkin lymphoma. In some embodiments, the cancer is a B-cell malignancy. In some embodiments, the B-cell malignancy is chronic lymphocytic leukemia (CLL), small lymphocytic lymphoma (SLL), diffuse large B-cell lymphoma (DLBCL), follicular lymphoma (FL), activated B-cell diffuse large B-cell lymphoma (ABC-DLBCL), germinal center diffuse large B-cell lymphoma (GCB DLBCL), primary mediastinal B-cell lymphoma (PMBL), Burkitt's lymphoma, immunoblastic large cell lymphoma, precursor B-lymphoblastic lymphoma, mantle cell lymphoma (MCL), B cell prolymphocytic leukemia, lymphoplasmacytic lymphoma, Waldenstrom macroglobulinemia, splenic marginal zone lymphoma, plasma cell myeloma, plasmacytoma, extranodal marginal zone B cell lymphoma, nodal marginal zone B cell lymphoma, mediastinal (thymic) large B cell lymphoma, intravascular large B cell lymphoma, primary effusion lymphoma, or lymphomatoid granulomatosis. In some embodiments, the cancer is a T cell malignancy. In some embodiments, the T cell malignancy is peripheral T cell lymphoma not otherwise specified (PTCL-NOS), anaplastic large cell lymphoma, angioimmunoblastic lymphoma, cutaneous T cell lymphoma, adult T cell leukemia/lymphoma (ATLL), blastic NK-cell lymphoma, enteropathy-type T cell lymphoma, hematosplenic gamma-delta T cell lymphoma, lymphoblastic lymphoma, nasal NK/T cell lymphomas, or treatment-related T cell lymphomas. In some embodiments, the subject can have multiple myeloma.

The subject can have a relapsed or refractory cancer.

The methods disclosed herein can further involve the administration of one or more additional agents to treat cancer, such as chemotherapeutic agents (e.g., Adriamycin, Cerubidine, Bleomycin, Alkeran, Velban, Oncovin, Fluorouracil, Thiotepa, Methotrexate, Bisantrene, Noantrone, Thiguanine, Cytaribine, Procarabizine), immuno-oncology agents (e.g., anti-PD-L1, anti-CTLA4, anti-PD-1, anti-CD47, anti-GD2), cellular therapies (e.g., CAR-T, T cell therapy, natural killer cell therapy, gamma delta T cell therapy), oncolytic viruses and the like.

Non-limiting examples of additional agents to treat cancer include acivicin, aclarubicin, acodazole hydrochloride, acronine, adozelesin, aldesleukin, altretamine, ambomycin, ametantrone acetate, aminoglutethimide, amsacrine, anastrozole, anthramycin, asparaginase, asperlin, azacitidine, azetepa, azotomycin, batimastat, benzodepa, bicalutamide, bisantrene hydrochloride, bisnafide dimesylate, bizelesin, bleomycin sulfate, brequinar sodium, bropirimine, busulfan, cactinomycin, calusterone, caracemide, carbetimer, carboplatin, carmustine, carubicin hydrochloride, carzelesin, cedefingol, chlorambucil, cirolemycin, cisplatin, cladribine, crisnatol mesylate, cyclophosphamide, cytarabine, dacarbazine, dactinomycin, daunorubicin hydrochloride, decitabine, dexormaplatin, dezaguanine, dezaguanine mesylate, diaziquone, docetaxel, doxorubicin, doxorubicin hydrochloride, droloxifene, droloxifene citrate, dromostanolone propionate, duazomycin, edatrexate, eflornithine hydrochloride, elsamitrucin, enloplatin, enpromate, epipropidine, epirubicin hydrochloride, erbulozole, esorubicin hydrochloride, estramustine, estramustine phosphate sodium, etanidazole, etoposide, etoposide phosphate, etoprine, fadrozole hydrochloride, fazarabine, fenretinide, floxuridine, fludarabine phosphate, fluorouracil, flurocitabine, fosquidone, fostriecin sodium, gemcitabine, gemcitabine hydrochloride, hydroxyurea, idarubicin hydrochloride, ifosfamide, ilmofosine, interleukin II (including recombinant interleukin II, or rIL2), interferon alpha-2a, interferon alpha-2b, interferon alpha-n1 interferon alpha-n3, interferon beta-Ia, interferon gamma-Ib, iproplatin, irinotecan hydrochloride, lanreotide acetate, letrozole, leuprolide acetate, liarozole hydrochloride, lometrexol sodium, lomustine, losoxantrone hydrochloride, masoprocol, maytansine, mechlorethamine hydrochloride, megestrol acetate, melengestrol acetate, melphalan, menogaril, mercaptopurine, methotrexate, methotrexate sodium, metoprine, meturedepa, mitindomide, mitocarcin, mitocromin, mitogillin, mitomalcin, mitomycin, mitosper, mitotane, mitoxantrone hydrochloride, mycophenolic acid, nocodazole, nogalamycin, ormaplatin, oxisuran, paclitaxel, pegaspargase, peliomycin, pentamustine, peplomycin sulfate, perfosfamide, pipobroman, piposulfan, piroxantrone hydrochloride, plicamycin, plomestane, porfimer sodium, porfiromycin, prednimustine, procarbazine hydrochloride, puromycin, puromycin hydrochloride, pyrazofurin, riboprine, rogletimide, safingol, safingol hydrochloride, semustine, simtrazene, sparfosate sodium, sparsomycin, spirogermanium hydrochloride, spiromustine, spiroplatin, streptonigrin, streptozocin, sulofenur, talisomycin, tecogalan sodium, tegafur, teloxantrone hydrochloride, temoporfin, teniposide, teroxirone, testolactone, thiamiprine, thioguanine, thiotepa, tiazofurin, tirapazamine, toremifene citrate, trestolone acetate, triciribine phosphate, trimetrexate, trimetrexate glucuronate, triptorelin, tubulozole hydrochloride, uracil mustard, uredepa, vapreotide, verteporfin, vinblastine sulfate, vincristine sulfate, vindesine, vindesine sulfate, vinepidine sulfate, vinglycinate sulfate, vinleurosine sulfate, vinorelbine tartrate, vinzolidine sulfate, vinzolidine sulfate, vorozole, zeniplatin, zinostatin, zorubicin hydrochloride.

The methods disclosed herein can further comprise administration of an anti-tumor antibody/drug conjugate. The anti-tumor antibody/drug conjugate can include, but not limited to, rituximab, cetuximab, trastuzumab, and pertuzumab, brentuximab vedotin, gemtuzumab ozogamicin, trastuzumab emtansine, inotuzumab ozogamicin, glembatumumab vedotin, lorvotuzumab mertansine, cantuzumab mertansine, or milatuzumab-doxorubicin.

The methods disclosed herein can further comprise administering an antiviral agent. Exemplary anti-viral agents include, but are not limited to, acyclovir, famciclovir, ganciclovir, penciclovir, valacyclovir, valganciclovir, idoxuridine, trifluridine, brivudine, cidofovir, docosanol, fomivirsen, foscarnet, tromantadine, imiquimod, podophyllotoxin, entecavir, lamivudine, telbivudine, clevudine, adefovir, tenofovir, boceprevir, telaprevir, pleconaril, arbidol, amantadine, rimantadine, oseltamivir, zanamivir, peramivir, inosine, interferon (e.g., Interferon alfa-2b, Peginterferon alfa-2a), ribavirin/taribavirin, abacavir, emtricitabine, lamivudine, didanosine, zidovudine, apricitabine, stampidine, elvucitabine, racivir, amdoxovir, stavudine, zalcitabine, tenofovir, efavirenz, nevirapine, etravirine, rilpivirine, loviride, delavirdine, atazanavir, fosamprenavir, lopinavir, darunavir, nelfmavir, ritonavir, saquinavir, tipranavir, amprenavir, indinavir, enfuvirtide, maraviroc, vicriviroc, PRO 140, ibalizumab, raltegravir, elvitegravir, bevirimat, and vivecon.

The compositions disclosed herein are typically administered systemically, for example by intravenous injection or intravenous infusion. Other routes of administration can be used, such as orally, parenterally, intravenous, intravenously, intra-articularly, intraperitoneally, intramuscularly, subcutaneously, intracavity, transdermally, intrahepatically, intracranially, nebulization/inhalation, by installation via bronchoscopy, or intratumorally.

The dosage regimen will be determined by the attending physician and other clinical factors. Dosages for any one patient depends on many factors, including the patient's size, body surface area, age, sex, the particular compound to be administered, time and route of administration, the kind of therapy, general health and other drugs being administered concurrently. An “effective dose” refers to amounts of the active ingredient that are sufficient to affect the course and the severity of the disease, leading to the reduction or remission of such pathology and may be determined using known methods.

### D. Methods of Preparing

The disclosure also relates to methods of preparing the adoptive cell therapy compositions disclosed herein. The method comprises obtaining a peripheral blood cell composition from a subject or from a donor subject. When the peripheral blood cell composition is from a donor subject, the donor subject is generally allogenic to a recipient subject or from a cell line or umbilical cord blood.

The peripheral blood cell composition can be a whole blood product or an apheresis product. The peripheral blood cell composition can be obtained using means known in the art, for example through venipuncture. The peripheral blood cell composition comprises both CD8+ T cells and CD4+ T cells. The peripheral blood cell composition can be obtained from human or non-human subjects. Preferentially, the peripheral blood cell composition is obtained from a human.

Immune cells from a donor subject can be mismatched to a recipient subject for at least one HLA Class II allele mismatch in the donor versus recipient (graft-versus-host) direction relative to the recipient subject. Alternatively, a donor can have at least one HLA class II allele mismatch relative to the recipient in the donor versus the recipient (graft-versus-host) direction and at least one HLA Class II allele match relative to the recipient. The HLA class II allele mismatch or match can be at HLA-DRB1, HLA-DQB1, or HLA-DPB1.

If the donor and recipient are ABO blood type incompatible and the allogenic leukocyte composition comprises a number of red blood cells, then making the adoptive cell therapy composition can further comprise reducing the number of red blood cells. “ABO blood type incompatible” as used herein refers to when the recipient has a major ABO red blood cell incompatibility against the donor, e.g., the recipient is blood type O, and the donor is blood type A, B, or AB, the recipient is type A and the donor is type B or AB, or the recipient is type B and the donor is type A or AB. The number of red blood cells can comprise less than or equal to about 50 ml in packed volume, e.g., less than or equal to about 50 ml in packed volume, preferably less than or equal to about 30 ml in packed volume, further “packed volume” should be defined, for example, centrifugation of the lymphocyte composition would result is a packed volume of 50 ml or less of red blood cells, a measured volume sample of the lymphocyte composition could also be screened to provide a proportionally representative volume of packed blood cells.

Mononuclear cells are then isolated from the peripheral blood cell composition, for example by Ficoll-Hypaque gradient separation. Next, the number of CD8+ cells in the leukocytes can be optionally depleted. The number of CD8+ cells in the leukocytes can be depleted by about 1 fold, about 2 fold, about 3 fold, about 4 fold, about 5 fold, about 6 fold, about 7 fold, about 8 fold, about 9 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 200 fold, about 300 fold, about 400 fold, about 500 fold, about 600 fold, about 700 fold, about 800 fold, about 900 fold, about 1,000 fold or greater relative to undepleted leukocytes.

The T cells are further modified to suppress BTK, ITK, or PI3Kδ, helios, blimp1, SOCS1, GATA3, IL-10, STAT3, TOX, CD25, foxp3, Ezh2, TGF-beta Receptor II, LAG-3, PD-1, TNF-alpha, or combinations thereof. Without wishing to be bound by theory or mechanism, the inventors believe that suppression of BTK, ITK, PI3Kδ, helios, blimp1, SOCS1, GATA3, IL-10, STAT3, TOX, CD25, foxp3, Ezh2, TGF-beta Receptor II, LAG-3, PD-1, TNF-alpha, or combinations thereof may promote naïve CD4+ T cells to differentiate to a state, such as type 1 (Th1) CD4+ T cells, that is favorable for helping effector cells of anti-tumor or anti-viral immunity, or prevent post-naïve CD4+ T cells from converting to cells with suboptimal helper activity for anti-tumor or anti-viral immunity. For example, a portion of the T cells may be preferentially differentiated to a CD4+ T cell sub-type, specifically Th1.

The method can comprise promoting differentiation of at least a portion of T cells toward Th1 CD4+ T cells. Suppression of BTK, ITK, PI3Kδ, helios, blimp1, SOCS1, GATA3, IL-10, STAT3, TOX, CD25, foxp3, Ezh2, TGF-beta Receptor II, LAG-3, PD-1, TNF-alpha, or combinations thereof can promote differentiation of a portion of T cells towards Th1 CD4+ T cells.

The method can comprise culturing the leukocytes in vitro.

The method of producing the adoptive cell therapy composition can further comprise stimulating antigen-specific lymphocytes in the composition with antigen-presenting cells pulsed with antigenic peptides.

The method of producing the allogeneic composition can further comprise adding one or more additional agents, such as a cytokine or antibodies.

The additional agent can be a cytokine. Exemplary cytokines that can be added include IL-2, IL-7, IL-12, IL-15, IL-18, IFNγ, IL-21, CCDC134, GM-CSF, or LYG1.

The additional agent can be an antibody. Exemplary antibodies include an anti-IL3 antibody, an anti-IL-4 antibody, an anti-CD3 antibody, an anti-CD200 antibody or an anti-CD28 antibody.

The additional agent can be an inhibitor. Exemplary inhibitors include inhibitors of MEK 1/2, ERK, p38, Cox-2, Pi13k, c512, setdb1, or Got1.

Other exemplary agents include, but are not limited to, receptor agonists (e.g., RAR alpha or TLR), transcription factors (e.g., T-bet and Tbx21), lipoarabinomannans, or lipomannans derived from BCG cell bodies.

### E. Definitions

Various terms relating to aspects of the description are used throughout the specification and claims. Such terms are to be given their ordinary meaning in the art unless otherwise indicated. Other specifically defined terms are to be construed in a manner consistent with the definitions provided herein. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

As used herein, the singular forms “a,” “an,” and “the” include plural forms unless the context clearly indicates otherwise. The terms “include,” “such as,” and the like are intended to convey inclusion without limitation, unless otherwise specifically indicated.

Unless otherwise indicated, the terms “at least,” “less than,” and “about,” or similar terms preceding a series of elements or a range are to be understood to refer to every element in the series or range. Those skilled in the art will recognize, or be able to ascertain using no more than routine experimentation, many equivalents to the specific embodiments of the invention described herein. Such equivalents are intended to be encompassed by the following claims.

The term “cancer” refers to the physiological condition in mammals in which a population of cells is characterized by uncontrolled proliferation, immortality, metastatic potential, rapid growth and proliferation rate and/or certain morphological features. Often cancers can be in the form of a tumor or mass, but may exist alone within the subject, or may circulate in the blood stream as independent cells, such a leukemic or lymphoma cells. The term cancer includes all types of cancers and metastases, including hematological malignancy, solid tumors, sarcomas, carcinomas and other solid and non-solid tumors. Examples of cancers include, but are not limited to, carcinoma, lymphoma, blastoma, sarcoma, and leukemia. More particular examples of such cancers include squamous cell cancer, small cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer (e.g., triple negative breast cancer), osteosarcoma, melanoma, colon cancer, colorectal cancer, endometrial (e.g., serous) or uterine cancer, salivary gland carcinoma, kidney cancer, liver cancer, prostate cancer, vulval cancer, thyroid cancer, hepatic carcinoma, and various types of head and neck cancers. Triple negative breast cancer refers to breast cancer that is negative for expression of the genes for estrogen receptor (ER), progesterone receptor (PR), and Her2/neu.

As used herein, the term “T cell exhaustion” refers to the progressive loss of effector function (loss of IL-2, TNF-α, and IFN-γ production, or failure to kill cells expressing the T cell's cognate antigen) and sustained expression of inhibitory receptors such as PD-1, T cell immunoglobulin domain, and mucin domain-containing protein 3 (Tim-3), CTLA-4, lymphocyte-activation gene 3 (LAG-3), and CD160 with a transcriptional program distinct from functional effector or memory T cells.

The term “subject” herein to refers to any animal, such as any mammal, including but not limited to, humans, non-human primates, rodents, and the like. In some embodiments, the mammal is a mouse. In some embodiments, the mammal is a human.

As used herein, the term “therapeutically effective amount” refers to an amount of a compound described herein (i.e., a composition) that is sufficient to achieve a desired pharmacological or physiological effect under the conditions of administration. For example, a “therapeutically effective amount” can be an amount that is sufficient to reduce the signs or symptoms of a disease or condition (e.g., a tumor). Those skilled in the art will appreciate that the therapeutic effects need not be complete or curative, as long as some benefit is provided to the subject. A therapeutically effective amount of a pharmaceutical composition can vary according to factors such as the disease state, age, sex, and weight of the individual, and the ability of the pharmaceutical composition to elicit a desired response in the individual. An ordinarily skilled clinician can determine appropriate amounts to administer to achieve the desired therapeutic benefit based on these and other considerations.

The term “neoantigen” as used herein refers to an antigen that has at least one alteration that makes it distinct from the corresponding parent antigen, e.g., via mutation in a tumor cell or post-translational modification specific to a tumor cell. A mutation can include a frameshift, indel, missense or nonsense substitution, splice site alteration, genomic rearrangement or gene fusion, or any genomic expression alteration giving rise to a neoantigen. A mutation can include a splice mutation. Post-translational modifications specific to a tumor cell can include aberrant phosphorylation. Post-translational modifications specific to a tumor cell can also include a proteasome-generated spliced antigen. See, Lipe et al., Science, 354(6310):354:358 (2016). In general, point mutations account for about 95% mutations in tumors and indels and frame-shift mutations account for the rest. See, Snyder et al., N Engl J Med., 371:2189-2199 (2014).

As used herein the term “tumor-specific neoantigen” is a neoantigen present in a subject's tumor cell or tissue, but not in the subject's normal cell or tissue.

### 1. EQUIVALENTS

It will be readily apparent to those skilled in the art that other suitable modifications and adaptions of the methods of the invention described herein are obvious and may be made using suitable equivalents without departing from the scope of the disclosure or the embodiments. Having now described certain compounds and methods in detail, the same will be more clearly understood by reference to the following examples, which are introduced for illustration only and not intended to be limiting.

### 2. EXAMPLES

The present invention is further described by the following examples, which are not intended to be limiting in any way.

Certain tumors are known to express tumor-associated antigens, like the E6 and E7 antigens of HPV type 16 (HPV16) or type 18 (HPV18). In other cases, neoantigens arise as malignant ones. Such neoantigens can be identified, for example, directly as described previously or by in silico prediction via a variety of methods as previously described. See, Lu et al., (2018), Mol Ther., 26(2):379-389; Lu et al., (2023), J. Immunother Cancer., 9(7):e002595; and Lang et al., (2022), Nat. Rev. Drug Discov., 21(4):261-282.

Once a tumor-associated antigen has been identified, T cell receptors (TCRs) responsive to such an antigen can be identified by a variety of methods as described elsewhere. See, Linnemann et al., (2013), 19(11):1534-1541. Such TCRs can then be expressed in lymphocytes, or other cell types, using methods of molecular engineering including, for example, CRISPR. See, e.g., Moosmann et al., (2021), STAR Protoc., 3(1):101031.

The following examples relate to the engineering of allogenic leukocytes or other allogeneic cells to modify the expression of certain regulatory genes and/or stimulatory molecules, including T cell receptors, and then using such allogeneic gene-modified cells to treat cancer.

### Example 1

### Validating Allogeneic TCR Transgenic Adoptive CD8+ Lymphocytes in Treatment of Known Antigen-Expressing Tumors

The objective of this experiment is to validate the treatment benefit resulting from infusion of an adoptive cell therapy composition composed of alloreactive CD8+ T cells with a TCR against a known tumor-associated antigen, with or without genetic modification to suppress SOCS1.

This experiment uses murine B16-F10 melanoma as a model for human tumors, including melanoma. B16-F10 expresses the mouse homologue for human gp100, pmel-17. The human gp100₂₅₋₃₃ epitope, KVPRNQDWL (SEQ ID NO:1), elicits a strong immune response, whereas its mouse homologue gp100₂₅₋₃₃, EGSRNQDWL (SEQ ID NO:2), is only weakly antigenic and does not elicit CD8+ T cell proliferation. The transgenic mouse strain Pmel-1 (Jackson Laboratory: B6.Cg-Thky1^{a}/Cy Tg(TcraTcrb)8Rest/J) expresses the Vα1Vβ13 T cell receptor, which specifically recognizes both H-2D^{b} restricted mouse and human gp100₂₅₋₃₃. Overwijk et al., (1998), J. Ex. Med., 188(2):277-286; Overwijk et al., (2003), J. Exp. Med., 198(4):569-580.

Pmel-1 is used as the platform to study treatment of B16-F10 tumors in C57BL/6 mice. Allogenic CD8+ T cells expressing Vα1Vβ13 will be generated by knocking the Vα1Vβ13 TCR into BALB/c primary CD8+ T cells (P-BALBs) as described previously. See, Moosmann et al., (2021), STAR Protoc., 3(1):101031; Schober et al., (2019), 3(12):974-984.

CD8+ T cells from donors will be isolated by negative selection using Miltenyi CD8a T Cell Isolation Kits (Miltenyi, cat, no. 130-095-236) as per Miltenyi protocol, SOCS1 KO will be achieved as described previously. Seki et al., (2018), J. Exp. Med., 215(3):985-997; Oh et al., (2019), Curr, Protoc. Immunol., 124(1):e69. T cells will be activated 1 day post-nucleofection with Miltenyi T Cell Activation/Expansion kits (cat. no. 130-093-627) and cultured in T cell medium composed of RPMI 1640 (Gibco, cat. no. 11875093), 10% FCS (HyClone, cat. no. SH30071.03), 2 mM L-alanyl-L-glutamine (GlutaMAX; Gibco, cat. no. 35050061), 1 mM sodium pyruvate (Gibco, cat. no. 11360070), 0.1 mM non-essential amino acids (Gibco, cat, no. 11140050), 55 μM 2-mercaptoethanol (Gibco, cat.no. 21985-023), 100 U/ml penicillin (PenStrep; Gibco, cat. no. 15140-122), 100 μg/ml streptomycin (PenStrep; Gibco, cat, no. 15140-122), 10 mM HEPES (Gibco, cat. no. 15630080), along with 5 ng/ml IL-7 and 5 ng/ml IL-15.

Table 1 shows the agents and treatment protocol for the study.
Recipients: C57BL/6J
Donors: (a) Syngeneic: Pmel-1, (b) Allogenic: P-BALB
Tumor infusion method: sub-cutaneous

TABLE 1
Agents and Treatment Protocol

| | | 50,000 | Cyclophos- | |
|---|---|---|---|---|
| | | B16-F10 | phamide 200 | 1 Million CD8+ |
| Group | N | S.C. | mg/kg IP d 14 | T Cells IV day 15 |
| 1 | 10 | + | − | − |
| 2 | 10 | + | + | − |
| 3 | 10 | + | + | Pmel-1 CD8+ T cells |
| 4 | 10 | + | + | P-BALB CD8+ T cells |
| 5 | 10 | + | + | Pmel-1 + SOCS1 KO |
| 6 | 10 | + | + | P-BALB + SOCS1 KO |

Mice will be followed for survival and tumor volume. Tumor volume will be measured twice weekly with calipers and volume calculated as V=(L×W²)/2.

### Example 2

### Validating Allogeneic TCR Transgenic Adoptive CD4+ Lymphocytes in Treatment of Known Antigen-Expressing Tumors

The objective of this experiment is to validate the treatment benefit resulting from infusion of an adoptive cell therapy composition composed of allogeneic CD4+ T cells with a TCR against a known tumor-associated antigen, with or without genetic modification to suppress SOCS1.

This experiment uses the murine B16-OVA M04 (B16-OVA) melanoma line (Sigma-Aldrich, cat. no. SCC420) as a model for human tumors, including melanoma. B16-OVA expresses chicken ovalbumin (OVA). The OT-II (Jackson Laboratory: B6.Cg-Tg(TcraTcrb)425Cbn/J) mouse cell line expresses the mouse αβ TCR specific for MHC class II I-A^{b} restricted OVA₃₂₃₋₃₃₉, and recognizes OVA in B16-OVA tumors.

OT-II is used as the platform to study treatment of 1316-OVA tumors in C57BL/6 mice. Allogeneic CD4+ T cells expressing the OT-II TCR will be generated by knocking the OT-II TCR into BALB/c primary CD4+ T cells (OT2-BALBs) as described previously. See, Moosmann et al., (2021), STAR Protoc., 3(1):101031; Schober et al., (2019), 3(12):974-984.

CD4+ T cells from donors will be isolated by negative selection using Miltenyi CD4+ T Cell Isolation Kits, mouse (Miltenyi, cat. no. 130-104-454) as per Miltenyi protocol. SOCS1 KO will be achieved as described previously. Oh et al., (2019), Curr. Protoc. Immunol., 124(1):e69. T cells will be activated 1 day post-nucleofection with Miltenyi T Cell Activation/Expansion kits (cat. no. 130-093-627) and cultured in T cell medium composed of RPMI 1640 (Gibco, cat. no. 11875093), 10% FCS (HyClone, cat. no. SH30071.03), 2 mM L-alanyl-L-glutamine (GlutaMAX; Gibco, cat. no. 35050061), 1 mM sodium pyruvate (Gibco, cat. no. 11360070), 0.1 mM non-essential amino acids (Gibco, cat. no. 11140050), 55 μM 2-mercaptoethanol (Gibco, cat. No. 21985-023), 100 U/ml penicillin (PenStrep; Gibco, cat. no. 15140-122), 100 μg/ml streptomycin (PenStrep; Gibco, cat, no. 15140-122), 10 mM HEPES (Gibco, cat. no. 15630080), along with 5 ng/ml IL-7 and 5 ng/ml IL-15.

Table 2 shows the agents and treatment protocol for the study.
Recipients: C57BL/6J
Donors: (a) Syngeneic: OT-II, (b) Allogenic: OT2-BALB
Tumor infusion method: sub-cutaneous

TABLE 2
Agents and Treatment Protocol

| | | 50,000 | Cyclophos | |
|---|---|---|---|---|
| | | B16-OVA | phamide 200 | 1 Million CD4+ T |
| Group | N | S.C. | mg/kg IP d 14 | Cells IV day 15 |
| 1 | 10 | + | − | − |
| 2 | 10 | + | + | − |
| 3 | 10 | + | + | OT-II CD4+ T cells |
| 4 | 10 | + | + | OT2-BALB CD4+ T cells |
| 5 | 10 | + | + | OT-II + SOCS1 KO |
| 6 | 10 | + | + | OT2-BALB + SOCS1 KO |

Mice will be followed for survival and tumor volume. Tumor volume will be measured twice weekly with calipers and volume calculated as V=(L×W²)/2.

### Example 3

### Validating Allogeneic Adoptive CAR-T Cells in Treatment of Known Antigen-Expressing Tumors

The objective of this experiment is to validate the treatment benefit resulting from infusion of an adoptive cell therapy composition composed of allogeneic CD8+ T cells with a TCR against a known tumor-associated target with or without genetic modification to suppress SOCS1.

This experiment uses the murine A20 lymphoma cell line (ATCC: cat. no. TIB-208) as a model for human cancers, including lymphoma. The A20 cell line is a BALB/c B cell lymphoma that expresses CD19. Chimeric Antigen Receptor (CAR) T cells specific for CD19 will be generated with a CD19scFv-CD28-4-1BB CAR construct and knocked in orthotopically to the TRAC locus, as previously described in both syngeneic BALB/cJ (BALB-CAR) and allogeneic C57BL/6J (B6-CAR) donor mice. See, Moosmann et al., (2021), STAR Protoc., 3(1):101031; Schober et al., (2019), 3(12):974-984.

T cells from donors will be isolated by negative selection using Miltenyi Pan T Cell Isolation Kit II (Miltenyi, cat. no. 130-095-130) as per Miltenyi protocol. SOCS1 KO will be achieved as described previously. Seki et al., (2018), J. Exp. Med., 215(3):985-997; Oh et al., (2019), Curr. Protoc. Immunol., 124(1):e69. T cells will be activated 1 day post-nucleofection with Miltenyi T Cell Activation/Expansion kits (cat, no. 130-093-627) and cultured in T cell medium composed of RPMI 1640 (Gibco, cat. no. 11875093), 10% FCS (HyClone, cat. no, SH30071.03), 2 mM L-alanyl-L-glutamine (GlutaMAX; Gibco, cat. no. 35050061), 1 mM sodium pyruvate (Gibco, cat. no. 11360070), 0.1 mM non-essential amino acids (Gibco, cat. no. 11140050), 55 μM 2-mercaptoethanol (Gibco, cat, No, 21985-023), 100 U/ml penicillin (PenStrep; Gibco, cat. no. 15140-122), 100 μg/ml streptomycin (PenStrep; Gibco, cat. no. 15140-122), 10 mM HEPES (Gibco, cat, no. 15630080), along with 5 ng/ml IL-7 and 5 ng/ml IL-15.

Table 3 shows the agents and treatment protocol for the study.
Recipients: BALB/cJ
Donors: (a) Syngeneic: CAR-BALB, (b) Allogenic: CAR-B6
Tumor infusion method: intravenous

TABLE 3
Agents and Treatment Protocol

| | | | Cyclophos- | |
|---|---|---|---|---|
| | | 10⁶ | phamide 200 | 1 Million CD4+ T |
| Group | N | A20 IV | mg/kg IP d 14 | Cells IV day 15 |
| 1 | 10 | + | − | − |
| 2 | 10 | + | + | − |
| 3 | 10 | + | + | BALB-CAR T cells |
| 4 | 10 | + | + | B6-CD19 CAR T cells |
| 5 | 10 | + | + | BALB-CD19 CAR + SOCS1 |
| | | | | KO |
| 6 | 10 | + | + | B6-CD19 CAR + SOCS1 KO |

Mice will be followed for survival and observed for signs of cytokine release syndrome (CRS) or other distress.

### Example 4

### Validating Allogeneic TCR Transgenic Adoptive CD4+ Lymphocytes in Treatment of Known Antigen-Expressing Infectious Disease

The objective of this experiment is to validate the treatment benefit resulting from infusion of an adoptive cell therapy composition composed of allogeneic CD4+ T cells with a TCR against a known infection-associated antigen, with or without genetic modification to suppress SOCS1.

This experiment uses lymphocytic choriomeningitis virus (LCMV) as a model for a variety of infections in humans, including LCMV. Infection of C57BL/6 mice with LCMV Clone 13 results in persistent infection. Oldstone et al., (2018), Proc. Natl. Acad. Sci USA, 115(33):E7814-E7823.

SMARTA-1 (SMARTA) is used as the platform to study treatment of LCMV infections in C57BL/6 mice. The SMARTA-1 (Jackson Laboratory: B6.Cg-Ptprc^{a} Pepc^{b} Tg(TcrLCMV)1Aox/PpmJ) mouse strain is a C57BL/6-congenic mouse with a rearranged T cell receptor (Vα2.3-JαDK1 and Vβ8.3-Jβ2.5) specific for peptide P13 of LCMV, GP₆₁₋₈₀. Allogenic CD4+ T cells expressing the SMARTA-1 TCR will be generated by knocking the SMARTA-1 TCR into BALB/c T cells (SMARTA-BALBs) as described previously. See, Moosmann et al., (2021), STAR Protoc., 3(1):101031; Schober et al., (2019), 3(12):974-984.

CD4+ T cells from donors will be isolated by negative selection using Miltenyi CD4+ T Cell Isolation Kits, mouse (Miltenyi, cat. no. 130-104-454) as per Miltenyi protocol. SOCS1 KO will be achieved as described previously. Seki et al., (2018), J. Exp. Med., 215(3):985-997; Oh et al., (2019), Curr. Protoc. Immunol., 124(1):e69. T cells will be activated 1 day post-nucleofection with Miltenyi T Cell Activation/Expansion kits (cat. no. 130-093-627) and cultured in T cell medium composed of RPMI 1640 (Gibco, cat, no. 11875093), 10% FCS (HyClone, cat. no. SH30071.03), 2 mM L-alanyl-L-glutamine (GlutaMAX; Gibco, cat. no. 35050061), 1 mM sodium pyruvate (Gibco, cat. no. 11360070), 0.1 mM non-essential amino acids (Gibco, cat. no. 11140050). 55 μM 2-mercaptoethanol (Gibco, cat. No. 21985-023), 100 U/ml penicillin (PenStrep; Gibco, cat. no. 15140-122), 100 μg/ml streptomycin (PenStrep; Gibco, cat. no. 15140-122), 10 mM HEPES (Gibco, cat. no. 15630080), along with 5 ng/ml IL-7 and 5 ng/ml IL-15.

Table 4 shows the agents and treatment protocol for the study.
Recipients: C57BL/6
Donors: (a) Syngeneic: SMARTA-1, (b) Allogenic: SMARTA-BALB
Tumor infusion method: intravenous

TABLE 4
Agents and Treatment Protocol

| | | | Cyclophos | |
|---|---|---|---|---|
| | | 2 × 10⁶ | phamide 200 | 1 Million CD4+ |
| Group | N | PFU IV | mg/kg IP d 14 | T Cells IV day 15 |
| 1 | 10 | + | − | − |
| 2 | 10 | + | − | SMARTA CD4+ T cells |
| 3 | 10 | + | − | SMARTA-BALB CD4+ T cells |
| 4 | 10 | + | − | SMARTA + SOCS1 KO |
| 5 | 10 | + | − | SMARTA-BALB + SOCS1 KO |

Mice will be followed for viral titer, infection clearance, and survival.

Although the invention has been described with reference to the above examples, it will be understood that modifications and variations are encompassed within the spirit and scope of the invention. Accordingly, the invention is limited only by the following claims.

1. An adoptive cell therapy composition comprising a population of isolated immune cells obtained from a subject,
wherein the immune cells are modified to suppress Bruton's tyrosine kinase (BTK), interleukin-2-inducible T cell kinase (ITK), delta isoform of phosphoinositide 3-kinase (PI3Kδ), helios, blimp1, SOCS1, GATA3, IL-10, STAT3, TOX, CD25, foxp3, Ezh2, TGF-beta Receptor II, LAG-3, PD-1, TNF-alpha, or combinations thereof,
wherein the immune cells are one or more T cells are T cells, natural killer (NK) cells, macrophages, or combinations thereof, and
wherein the immune cells are optionally depleted of CD8+ T cells by about 10-fold or greater relative to un-depleted leukocytes.

2. The composition of claim 1, wherein the adoptive cell therapy composition comprises one or more T cells, natural killer (NK) cells, or macrophages.

3. The composition of claim 1, wherein the adoptive cell therapy composition comprises tumor infiltrating lymphocytes (TILs), chimeric receptor T cells (CAR-T), chimeric NK cells (CAR-NK), or chimeric macrophages (CAR macrophages), or T-cell receptor (“TCR”)-transduced T cells.

4-5. (canceled)

6. The composition of claim 1, wherein the adoptive cell therapy comprises an immune cell that expresses an antigen binding protein.

7. The composition of claim 6, wherein the antigen binding protein has specificity for an antigen selected from the group consisting of CD19, CD123, CD22, CD30, CD171, CS-1, CLL-1 (CLECL1), CD33, EGFRvIII, GD2, GD3, BCMA, Tn Ag, PSMA, ROR1, FLT3, TAG72, CD38, CD44v6, CEA, EPCAM, B7H3, KIT, IL-13Ra2, Mesothelin, IL-11Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, FAP, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, TSHR, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, legumain, HPV E6, E7, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, fibronectin EDB (EDB-FN), 5T4 oncofetal antigen, and IGLL1.

8. (canceled)

9. The adoptive cell therapy composition of claim 1 or 2, wherein the immune cells are biased toward Th1 CD4+ T cell differentiation by inhibition of BTK, ITK, PI3Kδ, Foxp3, GATA3, STAT3, CD25, or Ezh2.

10-20. (canceled)

21. The adoptive cell therapy composition of claim 1 or 2, wherein BTK, ITK and PI3Kδ, helios, blimp1, SOCS1, GATA3, IL-10, STAT3, TOX, CD25, TGF-beta Receptor II, LAG-3, PD-1, TNF-alpha, foxp3, or Ezh2 are inhibited with an inhibitor or by genetic modification.

22. (canceled)

23. The adoptive cell therapy composition of claim 1 or 2, wherein BTK, ITK, PI3Kδ, Helios, Blimp1, SOCS1, Foxp3, GATA3, IL-10, TGF-beta Receptor II, LAG-3, PD-1, TNF-alpha, STAT3, Ezh2, CD25, or TOX are inhibited by deleting the BTK gene, the ITK gene, the PI3Kδ gene, the Helios gene, the Blimp1 gene, the SOCS1 gene, the Foxp3 gene, the TGF-beta Receptor II gene, the LAG-3 gene, the PD-1 gene, the TNF-alpha gene, the GATA3 gene, the IL-10 gene, the STAT3 gene, the Ezh2 gene, the CD25 gene, or the TOX gene from the genome using CRISPR or TALEN.

24-25. (canceled)

26. The adoptive cell therapy composition of claim 1 or 2, wherein differentiation of the T cells into T regulatory cells is attenuated through inhibition of PI3Kδ, Foxp3, CD25, or Ezh2 with a PI3Kδ inhibitor, a Foxp3 inhibitor, a CD25 inhibitor, or a Ezh2 inhibitor or by genetic modification.

27-29. (canceled)

30. The adoptive cell therapy composition of claim 1 or 2, wherein the isolated immune cells are obtained from a donor subject.

31. The adoptive cell therapy composition of claim 30, wherein the donor subject is mismatched to a recipient subject for at least one human leukocyte antigen (HLA) Class II allele in the donor versus recipient (graft-versus-host) direction relative to the recipient subject.

32. The adoptive cell therapy composition of claim 30, wherein the donor CD4+ T cells have been stimulated in vivo or ex vivo by an antigen present in a recipient subject, and the donor subject comprises at least one HLA Class II allele match relative to the recipient.

33. The adoptive cell therapy composition of claim 30, wherein the immune cells obtained from the donor subject (i) are mismatched to a recipient subject for at least one HLA Class II allele mismatch in the donor versus recipient (graft-versus-host) direction relative to the recipient subject and (ii) the donor CD4+ T cells have been stimulated in vivo or ex vivo by an antigen present in a recipient subject, and the donor subject comprises at least one human leukocyte HLA Class II allele match relative to the recipient.

34. The adoptive cell therapy composition of claim 33, wherein the HLA Class II match is an HLA-DRB1 allele, an HLA-DQB1 allele, or an HLA-DPB1 allele.

35. The adoptive cell therapy composition of claim 1, wherein the isolated immune cells are autologous immune cells.
